SEQUENCE LISTING (47 sequences)

SEQ ID NO: 1 - DNA, 44 bp, Saccharomyces cerevisiae
acaaaagctcttccgcttttggcaatgtcaacagtacccttagt

SEQ ID NO: 2 - DNA, 34 bp, Saccharomyces cerevisiae
tatatagctcttcgcgcttcctcgctcactgact

SEQ ID NO: 3 - DNA, 39 bp, Saccharomyces cerevisiae
cagtgagctcttcagcgctagagcgcccaatacgcaaac

SEQ ID NO: 4 - DNA, 35 bp, Saccharomyces cerevisiae
cgccaggctcttctcaaatttcggggacacttcct

SEQ ID NO: 5 - DNA, 33 bp, Saccharomyces cerevisiae
agtgtcgctcttctttgatcatatgcgccagcg

SEQ ID NO: 6 - DNA, 37 bp, Saccharomyces cerevisiae
tatatagctcttcgagcgacaaagattttgttatcgg

SEQ ID NO: 7 - DNA, 51 bp, Saccharomyces cerevisiae
ggggacaagtttgtacaaaaaagcaggcttaaagggaacaaaagctggagc

SEQ ID NO: 8 - DNA, 48 bp, Saccharomyces cerevisiae
ggggaccactttgtacaagaaagctgggtaaaagccttcgagcgtccc

SEQ ID NO: 9 - DNA, 46 bp, Saccharomyces cerevisiae
gacaagctgtgacgctcttcggatcatttatctttcactgcggaga

SEQ ID NO: 10 - DNA, 53 bp, Saccharomyces cerevisiae
gtgccagctgcgctcttcggttttagagctagaaatagcaagttaaaataagg

SEQ ID NO: 11 - DNA, 56 bp, Saccharomyces cerevisiae
ctccgcagtgaaagataaatgatccgaagagcgtcacagcttgtctgtaagcggat

SEQ ID NO: 12 - DNA, 42 bp, Saccharomyces cerevisiae
atttctagctctaaaaccgaagagcgcagctggcacgacagg

SEQ ID NO: 13 - DNA, 47 bp, Saccharomyces cerevisiae
accaagttgctgcaaaagtcgatcatttatctttcactgcggagaag

SEQ ID NO: 14 - DNA, 54 bp, Saccharomyces cerevisiae
gacttttgcagcaacttggtgttttagagctagaaatagcaagttaaaataagg

SEQ ID NO: 15 - DNA, 46 bp, Saccharomyces cerevisiae
tactatggcaactcctaacgatcatttatctttcactgcggagaag

SEQ ID NO: 16 - DNA, 53 bp, Saccharomyces cerevisiae
gttaggagttgccatagtagttttagagctagaaatagcaagttaaaataagg

SEQ ID NO: 17 - DNA, 22 bp, Saccharomyces cerevisiae
atcgattgaggggggcttgatg

SEQ ID NO: 18 - DNA, 22 bp, Saccharomyces cerevisiae
aaccatcaagcccccctcaatc

SEQ ID NO: 19 - DNA, 58 bp, Saccharomyces cerevisiae
ttgtcgatattcatcttattaaattcttaatactatggcaactcctaacgaaactatt

SEQ ID NO: 20 - DNA, 58 bp, Saccharomyces cerevisiae
aatagtttcgttaggagttgccatagtattaagaatttaataagatgaatatcgacaa

SEQ ID NO: 21 - DNA, 57 bp, Saccharomyces cerevisiae
attggccttgttgttggtcagctaaaaccaagtttttaaaagtccacaaggctaaaa

SEQ ID NO: 22 - DNA, 57 bp, Saccharomyces cerevisiae
ttttagccttgtggacttttaaaaacttggttttagctgaccaacaacaaggccaat

SEQ ID NO: 23 - DNA, 59 bp, Saccharomyces cerevisiae
attgcttgtgtcgcccgtgatgagcgtttaaatcaagcccccctcaatcacctgaaaca

SEQ ID NO: 24 - DNA, 59 bp, Saccharomyces cerevisiae
tgtttcaggtgattgaggggggcttgatttaaacgctcatcacgggcgacacaagcaat

SEQ ID NO: 25 - DNA, 23 bp, Saccharomyces cerevisiae
ggtgccagaaaatagaaggaaac

SEQ ID NO: 26 - DNA, 23 bp, Saccharomyces cerevisiae
gttgtgagcgataaaattcccag

SEQ ID NO: 27 - DNA, 20 bp, Saccharomyces cerevisiae
ttcatttgcgggtgtcgatg

SEQ ID NO: 28 - DNA, 20 bp, Saccharomyces cerevisiae
ggaacatcgtgaccaccttc

SEQ ID NO: 29 - DNA, 21 bp, Saccharomyces cerevisiae
tccagacggaaggctatcatg

SEQ ID NO: 30 - DNA, 21 bp, Saccharomyces cerevisiae
atgttgccatttttgtcctcc

SEQ ID NO: 31 - DNA, 39 bp, Saccharomyces cerevisiae
tatatagcggccgcatgccaactttgtacaaaaaagcag

SEQ ID NO: 32 - DNA, 57 bp, Saccharomyces cerevisiae
caatttcaagagagcataattagtacactgtccgcttcctgctttcactttgcgtag

SEQ ID NO: 33 - DNA, 60 bp, Saccharomyces cerevisiae
tgctctcttgaaattggctggagatgttgagagcaaccctggacctatggaattatcttg

SEQ ID NO: 34 - DNA, 37 bp, Saccharomyces cerevisiae
tatataagatcttgtacaagaaagctgggtattatgt

SEQ ID NO: 35 - DNA, 38 bp, Saccharomyces cerevisiae
tatatagtcgacatgacttcttccaattccgatttagt

SEQ ID NO: 36 - DNA, 55 bp, Saccharomyces cerevisiae
cgtcaccgcatgttagcagacttcctctgccctcccagacatccctaaggtagcg

SEQ ID NO: 37 - DNA, 54 bp, Saccharomyces cerevisiae
ctgctaacatgcggtgacgtcgaggagaatcctggcccaatggagcctaatttc

SEQ ID NO: 38 - DNA, 36 bp, Saccharomyces cerevisiae
tatatactcgagttaagctttgtgtggataaaggcg

SEQ ID NO: 39 - DNA, 76 bp, Saccharomyces cerevisiae
aatggttttgtcgatattcatcttattaaattctaccttactatggcaactcctaacgaaactattatttggtgaa

SEQ ID NO: 40 - DNA, 58 bp, Saccharomyces cerevisiae; identical to SEQ ID NO: 19

SEQ ID NO: 41 - DNA, 75 bp, Saccharomyces cerevisiae
aatggttttgtcgatattcatcttattaaattcttaatactatggcaactcctaacgaaactattatttggtgaa

SEQ ID NO: 42 - DNA, 76 bp, Saccharomyces cerevisiae
tattgccattggccttgttgttggtcagcgccaaccaagttgctgcaaaagtccacaaggctaaaatttataaaca

SEQ ID NO: 43 - DNA, 57 bp, Saccharomyces cerevisiae; identical to SEQ ID NO: 21

SEQ ID NO: 44 - DNA, 74 bp, Saccharomyces cerevisiae
tattgccattggccttgttgttggtcagctaaaaccaagtttttaaaagtccacaaggctaaaatttataaaca

SEQ ID NO: 45 - DNA, 75 bp, Saccharomyces cerevisiae
ctgacgaattgcttgtgtcgcccgtgatgagcgccacatcaagcccccctcaatcacctgaaacatccatcttag

SEQ ID NO: 46 - DNA, 59 bp, Saccharomyces cerevisiae; identical to SEQ ID NO: 23

SEQ ID NO: 47 - DNA, 76 bp, Saccharomyces cerevisiae
ctgacgaattgcttgtgtcgcccgtgatgagcgtttaaatcaagcccccctcaatcacctgaaacatccatcttag

(19) United States
(12) Patent Application Publication
(10) Pub. No.: US 2020/0385762 A1
(43) Pub. Date: Dec. 10, 2020

(54) Means and Methods for the Production of Terpenoids

(71) Applicants: VIB VZW, Gent (BE); UNIVERSITEIT GENT, Gent (BE)

(72) Inventors: Alain Goossens, Lokeren (BE); Philipp Arendt, Gent (BE); Nico Callewaert, Nevele (BE)

(21) Appl. No.: 16/463,932

(22) PCT Filed: Nov. 27, 2017
(86) PCT No.: PCT/EP2017/080542

§ 371 Date: May 24, 2019

(30) Foreign Application Priority Data
Nov. 28, 2016 (EP) 16200873.4

(51) Int. Cl. C12P 5/00; C12N 1/18; C12N 9/12; C12N 9/16

(52) U.S. Cl. CPC C12P 5/007; C12N 9/16; C12N 9/1205; C12N 1/18

(57) ABSTRACT

The present application relates to the field of terpenoid production technologies, particularly to production technologies using recombinant eukaryotic cells, and the improvement thereof. In particular, the present invention relates to recombinant eukaryotic cells capable of producing increased yields of terpenoids. Accordingly, the invention provides eukaryotic cells wherein intracellular membrane proliferation is affected and as such stimulated. The invention as well provides methods for the production of said cells.

### FIELD OF THE INVENTION

The present application relates to the field of terpenoid production technologies, particularly to production technologies using recombinant eukaryotic cells, and the improvement thereof. In particular, the present invention relates to recombinant eukaryotic cells capable of producing increased yields of terpenoids. Accordingly, the invention provides eukaryotic cells wherein intracellular membrane proliferation is affected and as such stimulated. The invention as well provides methods for the production of said cells.

### BACKGROUND

Terpenoids are molecules derived from a five-carbon isoprene unit that are assembled and modified in different ways and have diverse activities. Their structures are given by terpenoid biosynthesis enzymes. A particular class of terpenoids are the saponins which are members of the triterpene subfamily of terpenoids and are synthesized in plants via the mevalonic acid (MVA) pathway. Analogous to the biosynthesis of the membrane steroids cholesterol and ergosterol, the first committed step in the biosynthesis of saponins is the cyclization of 2,3-oxidosqualene by action of oxidosqualene cyclases (OSCs) to a variety of tri-, tetra-, or pentacyclic structures. In contrast to membrane steroids, the triterpene skeletons subsequently undergo various functionalizations such as cytochrome P450-mediated oxidation. These modifications serve as anchor points for subsequent conjugations such as glycosylation through UDP-dependent glycosyltransferases (UGTs), thereby rendering the highly apolar compounds amphipathic (Seki, H. et al., Plant Cell Physiol., 2015; Thimmappa, R. et al., Annu. Rev. Plant Biol., 2014).

Many saponins and their aglycones, sapogenins, exhibit valuable pharmacological properties. However, many triterpenoids of pharmacological interest accumulate only in little amounts in their natural hosts and their purification is challenging due to the complex plant metabolome with hundreds of compounds with similar chemical properties. Furthermore, due to the complex structure and chirality of many triterpenoids, their synthesis by chemical means is not trivial. An attractive alternative is the production in heterologous microbial hosts as these are easy to engineer, inexpensive to grow and generally display a simpler chemical complexity of metabolites. Especially the budding yeast Saccharomyces cerevisiae has emerged as the work horse for terpenoid engineering and a semi-synthetic yeast platform for the synthesis of the important anti-malarial drug artemisinin is currently the flag ship of metabolic engineering (Ro, D. K. et al., Nature, 2006; Paddon, C. J. et al., Nature, 2013; Peplow, M., Nature, 2013). S. cerevisiae is especially interesting for the synthesis of triterpenoids because as a eukaryote it possesses an endoplasmic reticulum (ER) that allows for the heterologous expression of membrane-localized cytochromes P450.

Classical metabolic engineering efforts for terpenoid-producing yeasts generally focus on boosting of the flux through the MVA pathway. This can be achieved by de-regulating the MVA gate keeper, HMG-CoA reductase (HMGR), which is subject to regulation on multiple levels (Burg, J. S. and Espenshade, P. J., Prog. Lipid Res., 2011). The two yeast HMGR isoforms underlie different means of regulation which can be overcome by over-expression of a truncated form of Hmg1p (tHMG1) or mutated Hmg2p (HMG2K6R). The overexpression of a mutated version of the sterol transcription factor UPC2 (upc2-1) furthermore leads to the up-regulation of most ERG genes and as such increases the metabolic flux through the MVA pathway (Davies, B. S. J. et al., Mol. Cell. Biol., 2005; Ro, D. K. et al., Nature, 2006; Westfall, P. J. et al., Pnas, 2012; Shiba, Y. et al., Metab. Eng., 2007). Finally, endogenous promoters of competing pathway branches such as squalene synthase (ERGS) for sesquiterpenes or lanosterol synthase (ERG7) for triterpenes can be replaced with repressible promoters such as the methionine-regulated PMET3 (Moses, T. et al., Proc. Natl. Acad. Sci. 2014; Ro, D. K. et al., Nature, 2006; Kirby, J. et al., FEBS J., 2008).

Despite these universal engineering strategies, the microbial production of most terpenoids is far from their optimal theoretical yields. Hence, it would be advantageous to develop new strategies such as targets for gene knockout for metabolic engineering programs.

### SUMMARY

It is an object of the invention to provide cells with higher production capacities of terpenoids, particularly triterpenoids, and methods of producing terpenoids in these cells. This is achieved by an increase in intracellular membrane proliferation, particularly through inhibition of the negative regulation of intracellular membrane proliferation. An inhibition of the negative regulation of intracellular membrane proliferation can be achieved by elevating phosphatidic acid (PA) content at the ER membrane. This, in turn, is achieved by inhibiting phosphatidic acid phosphatase and/or upregulation of diacylglycerol kinase activity, which results in expansion of the intracellular membrane.

When we disrupted the phosphatidic acid phosphatase-encoding PAH1 through CRISPR/Cas9, the intracellular membrane dramatically expanded while the cells remained viable. Surprisingly, we found an impressing increase of the production of recombinant triterpene biosynthesis enzymes. For example, expression of Glycyrrhiza glabra β-amyrin synthase (GgbAS) in the engineered yeast unexpectedly boosted the production of the oleanane-type sapogenin β-amyrin eightfold compared to the wild-type strain. Co-expression of the genes of the medicagenic acid pathway of Medicago truncatula, CYP716A12, CYP72A68, CYP72A67 together with MtCPR1, resulted in the sixfold increase of medicagenic acid production. Further pathway engineering through expression of UGT73F3 resulted in an even 16-fold increase in the production of medicagenic-28-O-glucoside in the pah1 strain compared to the wild-type. A positive effect of pah1 could also be observed for the production of other terpenoids depending on intracellular membrane-associated enzymes for their biosynthesis, such as the sesquiterpenoid artemisinic acid, which increased twofold. Also indirectly affecting PAH1 activity by treating cells with chemicals or by reducing the expression of PAH1 regulators resulted in increased production of terpenoids.

This is the first report on pathway engineering in yeast through engineering of the subcellular morphology rather than alteration of metabolic fluxes and also the highest reported unexpected effect of a single gene knockout for the production of heterologous terpenoids.

Thus, according to a first aspect, methods of enhancing production of terpenoids in a recombinant eukaryotic cell are provided, which entail that a recombinant eukaryotic cell deficient in expression and/or activity of an endogenous phosphatidic acid phosphatase, and/or overexpressing a diacylglycerol kinase is provided, wherein the recombinant cell comprises at least one chimeric gene construct comprising a promoter active in said recombinant eukaryotic cell operably fused to a nucleic acid sequence encoding the terpenoid biosynthesis enzyme and wherein the cell is maintained in conditions suitable for producing said terpenoid. Afterwards, the terpenoid or terpenoids of interest can then be recovered from the cell. Typically, the nucleic acid sequence encoding the terpenoid biosynthesis enzyme to be produced is an exogenous nucleic acid sequence or an endogenous nucleic acid sequence under control of an exogenous promoter. The terpenoid biosynthesis enzyme may be expressed constitutively or in an inducible way. Accordingly, the promoter may be a constitutive or inducible promoter. In particular embodiments, said terpenoid biosynthesis enzyme is plant derived or originates from plants.

According to particular embodiments, the endogenous phosphatidic acid phosphatase is PAH1 or a homolog thereof. According to alternative, but non-exclusive embodiments, the cell is deficient in expression and/or activity of the endogenous phosphatidic acid phosphatase through disruption of the endogenous phosphatidic acid phosphatase gene at nucleic acid level. Alternatively, the cell is deficient in expression and/or activity through an inhibitory RNA directed to the endogenous phosphatidic acid phosphatase gene transcript. Using for instance cells according to this latter embodiment, the deficiency of the expression and/or activity of the endogenous phosphatidic acid phosphatase may be inducible, which is envisaged in particular embodiments. According to yet alternative but non-exclusive embodiments, the cell is deficient in expression and/or activity of the endogenous phosphatidic acid phosphatase through disruption of at least one of the PAH1 regulatory complexes, i.e. the Ino2p/Ino4p/Opi1p regulatory circuit and the transcription factors Gis1p and Rph1p which bind to different positions of the PAH1 promoter and as such induce gene expression. According to yet alternative but non-exclusive embodiments, the cell is deficient in endogenous phosphatidic acid phosphatase activity through disruption of the PAH1 activating Nem1/Spo7 phosphatase complex or through treatment of said cell with phosphatidic acid phosphatase inhibitors such as propranolol, sphingosine, sphinganine, rutin, kaempferol, N-ethylmaleimide and bromoenol lactone.

According to other particular embodiments, the diacylglycerol kinase that is overexpressed is DGK1 or a homolog thereof. The diacylglycerol kinase that is overexpressed may be an endogenous diacylglycerol kinase or an exogenous diacylglycerol kinase. Typically, although not necessarily, the promoter driving the diacylglycerol kinase expression is an exogenous promoter. Overexpression of the diacylglycerol kinase may be constitutive or inducible. Likewise, the promoters driving the diacylglycerol kinase expression may be constitutive or inducible promoters.

According to yet other particular embodiments, the terpenoids that are produced in the eukaryotic cells described herein depend on intracellular membrane-associated enzymes for their biosynthesis. According to further particular embodiments, the terpenoids that are produced in the eukaryotic cells described herein depend on non-intracellular membrane-associated enzymes for their biosynthesis. According to other particular embodiments, the terpenoids that are produced in the eukaryotic cells described herein depend on both intracellular and non-intracellular membrane-associated enzymes for their biosynthesis. According to further particular embodiments, the terpenoid is selected from hemiterpenoids, monoterpenoids, sesquiterpenoids, diterpenoids, sesterpenoids, triterpenoids, tetraterpenoids, polyterpenoids or glycosides thereof. According to yet further particular embodiments, the terpenoid is beta-amyrin, an oleanane-type saponin, taxadiene or artemisinic acid. According to specific embodiments, more than one, i.e. two or more different terpenoids may be produced simultaneously.

According to other envisaged embodiments, the eukaryotic cells used for terpenoid production are yeast cells. According to even more particular embodiments, the yeast cells are from species of the genus Saccharomyces, such as Saccharomyces cerevisiae. Typically, the terpenoid that is produced in a yeast cell will be isolated (or possibly secreted) from the cell.

According to alternative particular embodiments, the eukaryotic cells are plant cells, particularly plant cell cultures. According to even more particular embodiments, the plant cells are from species of the genus Nicotiana such as Nicotiana tobacco, most particularly of Nicotiana benthamiana. According to yet further alternative embodiments, the eukaryotic cells are mammalian cells, most particularly Hek293 cells, such as Hek293S cells.

According to specific embodiments, the methods of terpenoid production also comprise the step of isolating the produced terpenoid. This typically involves recovery of the material wherein the terpenoid is present (e.g. a cell lysate or specific fraction thereof, the medium wherein the terpenoid is secreted) and subsequent purification of the terpenoid. Means that can be employed to this end are known to the skilled person.

According to a further aspect, recombinant eukaryotic cells with increased intracellular membrane proliferation compared to a control cell are provided herein that comprise at least one chimeric gene construct comprising a promoter active in said recombinant cell operably linked to a nucleotide sequence encoding a terpenoid biosynthesis enzyme to be expressed by the cell. In particular embodiments, said recombinant eukaryotic cells are deficient in expression and/or activity of an endogenous phosphatidic acid phosphatase and/or overexpress a diacylglycerol kinase.

According to particular embodiments, the eukaryotic cells are yeast cells. According to even more particular embodiments, the yeast cells are from species of the genus Saccharomyces, such as Saccharomyces cerevisiae.

As described for the methods above, in the eukaryotic cells the endogenous phosphatidic acid phosphatase is particularly envisaged to be PAH1 or a homolog thereof and/or the diacylglycerol kinase particularly is DGK1 or a homolog thereof.

According to further embodiments, the use of these cells for the production of terpenoids is provided herein. Also, the use of PAH1 inhibitors is provided for the increased production of terpenoids in a eukaryotic cell, wherein said PAH1 inhibitor is selected from the list consisting of propranolol, sphingosine, sphinganine, rutin, kaempferol, N-ethylmaleimide and bromoenol lactone.

The terpenoids that are produced in the eukaryotic cells described herein depend on intracellular membrane-associated enzymes for their biosynthesis. According to further embodiments, the terpenoids that are produced in the eukaryotic cells described herein depend on non-intracellular membrane-associated enzymes for their biosynthesis. According to other particular embodiments, the terpenoids that are produced in the eukaryotic cells described herein depend on both intracellular and non-intracellular membrane-associated enzymes for their biosynthesis.

According to further particular embodiments, the terpenoid is selected from hemiterpenoids, monoterpenoids, sesquiterpenoids, diterpenoids, sesterpenoids, triterpenoids, tetraterpenoids, polyterpenoids or glycosides thereof. According to yet further particular embodiments, the terpenoid is beta-amyrin, an oleanane-type saponin, taxadiene or artemisinic acid.

According to particularly envisaged embodiments, a cell culture of the recombinant eukaryotic cells as described herein is provided.

### BRIEF DESCRIPTION OF THE FIGURES

FIG. 1: Generation of BY4742 knockout strains using CRISPR/Cas9.

Knockout of HRD1, PEP4, and PAH1 in BY4742 by HR-mediated CRISPR events. Mismatches compared to the respective gene loci are indicated with red boxes.

FIG. 2: Relative production of β-amyrin in different yeast knockout strains.

(A) The cyclization of 2,3-oxidosqualene to β-amyrin is the first committed step in the biosynthesis of oleanane-type sapogenins and is catalyzed by β-amyrin synthases such as GgbAS from Glycyrrhiza glabra. The main fungal membrane steroid, ergosterol, is synthesized via the cyclization of oxidosqualene to lanosterol. Multiple enzymatic conversions are indicated by a dashed arrow. (B) Relative production levels of ergosterol and β-amyrin in wildtype and knockouts. Values are average of four independent cultures ±SE, *p<0.05, **p<0.01, ***p<0.001 relative to WT.

FIG. 3: Effect of PEP4 and PAH1 knockouts on the production of medicagenic acid.

(A) Metabolic pathway for the production of medicagenic acid through oxidation of β-amyrin at positions C28, C23 and C2 by CYP716A12, CYP72A67, and CYP72A68. (B) Production levels of medicagenic acid and intermediates in different genotypic backgrounds relative to wildtype. Values are average of five independent cultures ±SE, *p<0.05, **p<0.01, ***p<0.001 relative to WT.

FIG. 4: Expression analysis of heterologous GgbAS and CYP716A12 in pep4 and pah1 strains.

(A) 30 μg of total protein extracts were separated by SDS-PAGE and analyzed via immunoblot (α-HA). (B) Coomassie-stained membrane after immunoblot analysis as loading control.

FIG. 5: Growth phenotype of pah1 cells compared to wild-type.

PAH1 knockout cells exhibit a more pronounced lag phase and do not reach the same final OD₆₀₀ after 170 h of cultivation. Average of 5 independent cultures ±SE.

FIG. 6: The production of various triterpenoid skeletons is increased in the ER-engineered pah1 strain.

(A+B) Relative accumulation of OSC products in culture medium after expression of CaDDS (A) and AtTHAS1 (B). (C+D) Production levels of various (oxidized) sapogenins after expression of the OSC-P450 pairs GgbAS+CYP93E9 (C) and AtLUP1+CYP716A83 (D). Values for wild-type are shown in black, for pah1 in gray and are average of three independent cultures ±SE, *p<0.05, **p<0.01, ***p<0.001 relative to WT.

FIG. 7: Production of oleanane-type saponins in WT and pah1 strains.

(A) M. truncatula UGT73F3 generates the 28-O-glucosides of oleanane-type sapogenins such as medicagenic acid. (B) Relative production levels of mono-glucosylated saponins in wildtype and pah1 strains. Values are average of four independent cultures ±SE, *p<0.05, **p<0.01, ***p<0.001 relative to WT.

FIG. 8: Production of the sesquiterpenoid artemisinic acid in WT and pah1 strains.

(A) The anti-malarial drug artemisinin can be semi-synthetically generated by microbial production of artemisinic acid through amorpha-4,11-diene synthase and CYP71AV1 with subsequent chemical conversion (Ro et al., 2006). (B) Relative accumulation of artemisinic acid in culture media of wild-type and pah1 strains. Values are mean of 4 independent cultures ±SE, *p<0.05, **p<0.01, ***p<0.001 relative to WT.

FIG. 9: Production of GPP through engineered Erg20p and conversion to geraniol.

FIG. 10: Production of taxadiene from GGPP.

FIG. 11: Effect of OPI1 KO on the production of β-amyrin.

Shown are relative production levels of β-amyrin in two different genotypic backgrounds (PA14: BY4742; TRP1-Δ0; pMET3::ERG7 and PA59: BY4742; TRP1-Δ0) relative to the respective wild-type (WT). Values are averages of five independent cultures ±SE. Significant differences (Student's t-test) after Bonferroni corrections: ***, p<0.001 relative to WT. For both genotypes, two separate opi1 CRISPR knockouts were made (opi1-a and opi1-b). All four strains have a consistently higher (2- to 3-fold) β-amyrin production compared to their relative wild-types.

FIG. 12: Regulation of Pah1p activity through dephosphorylation by the Nem1p/Spo7p heterodimer (Dubots, E. et al., PloS one, 2014).

FIG. 13: Effect of propranolol on the production of CYP716A12.

Increased CYP716A12 protein levels in pah1 KO background can be mimicked by adding propranolol to the medium. Prop, propranolol.

### DETAILED DESCRIPTION

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4th ed., Cold Spring Harbor Press, Plainsview, N.Y. (2012); and Ausubel et al., Current Protocols in Molecular Biology (Supplement 114), John Wiley & Sons, New York (2016), for definitions and terms of the art. The definitions provided herein should not be construed to have a scope less than understood by a person of ordinary skill in the art.

A “eukaryotic cell” as used herein is a cell containing a nucleus and an endoplasmic reticulum or ER, which is involved in protein transport and maturation. The term eukaryotic cell as used herein refers to a recombinant eukaryotic cell wherein intracellular membrane proliferation is increased. Said increase of intracellular membrane proliferation of the recombinant eukaryotic cell can for instance be determined microscopically. Confocal microscopy could be used in combination with an intracellular membrane marker. Additionally and non-limiting, transmission electron microscopy (TEM) has a high resolution and as such gives direct proof of intracellular membrane morphology. An increase of intracellular membrane proliferation is typically induced by stimulation and determined by comparison with a control cell.

A “recombinant eukaryotic cell”, as used herein, refers to a cell produced by recombinant methods, e.g. recombinant DNA technology. For the purposes of the invention, “recombinant”, “transgene” or “transgenic” means with regard to, for example, a protein, a nucleic acid sequence, an expression cassette, gene construct or a vector comprising said nucleic acid sequence or a cell transformed with nucleic acid sequences, expression cassettes or vectors, all those constructions brought about by recombinant methods in which either (a) the nucleic acid sequences encoding proteins useful in the methods of the invention, or (b) genetic control sequence(s) which is operably linked with the nucleic acid sequence according to the invention, for example a promoter, or (c) a) and b) are not located in their natural genetic environment or have been modified by recombinant methods, it being possible for the modification to take the form of, for example, a substitution, addition, deletion, inversion or insertion of one or more nucleotide residues. The natural genetic environment is understood as meaning the natural genomic or chromosomal locus in the original cell or the presence in a genomic library.

In one embodiment, said recombinantly eukaryotic cell is a man-made or non-naturally occurring eukaryotic cell.

The term “intracellular membrane” as used herein refers to an intracellular, interconnected network of phospholipids. According to the invention, the fatty acid flux of this intracellular membrane is dysregulated away from triglycerides and into phospholipids. The endoplasmic reticulum can be seen as a typical and non-limiting example of an intracellular membrane according to the invention. “Proliferation” in the context of intracellular membrane proliferation means the growth or spread or increase or expansion or development of the intracellular membrane compartment. Throughout the application, “an increase of the intracellular membrane proliferation” is equivalent as “an increase of the intracellular membrane compartment development” or “an increase of the intracellular membrane compartment expansion”.

The term “control cell” refers to a comparable eukaryotic cell wherein no modifications have been made in order to stimulate intracellular membrane proliferation.

With a “chimeric gene” or “chimeric construct” or “chimeric gene construct” is meant a recombinant nucleic acid sequence in which an expressible promoter or regulatory nucleic acid sequence is operatively linked to, or associated with, a nucleic acid sequence or DNA region that codes for an mRNA, such that the regulatory nucleic acid sequence is able to regulate transcription or expression of the associated nucleic acid coding sequence. The regulatory nucleic acid sequence or promoter of the chimeric gene is not operatively linked to the associated nucleic acid sequence as found in nature, hence is heterologous to the coding sequence of the DNA region operably linked to. The term “operatively” or “operably” linked or fused as used herein refers to a functional linkage between the expressible promoter sequence and the DNA region or gene of interest, such that the promoter sequence is able to initiate transcription of the gene of interest, and refers to a functional linkage between the gene of interest and the transcription terminating sequence to assure adequate termination of transcription in eukaryotic cells. In the present invention an “expressible promoter for said eukaryotic cell” comprises regulatory elements, which mediate the expression of a coding sequence segment in said eukaryotic cells. For expression in yeast for instance, the nucleic acid molecule must be linked operably to or comprise a suitable promoter which expresses the gene at the right point in time and with the required spatial expression pattern in yeast cells. The chimeric gene construct(s) can be part of a vector that comprises multiple chimeric gene constructs or multiple genes, such as a selectable marker gene. Selectable marker genes may be used to identify transformed cells or tissues. The chimeric gene or chimeric genes to be expressed are preferably cloned into a vector, or recombinant vector, which is suitable for transforming the eukaryotic cell, or which is suitable to transform a bacterium mediating transformation, such as Agrobacterium tumefaciens, for example pBin19 (Bevan, M. W. et al, Nucl. Acids Res., 1984), mediating plant cell transformation.

The terms “regulatory element”, “control sequence” and “promoter” or “promoter region of a gene” are all used interchangeably herein and are to be taken in a broad context to refer to regulatory nucleic acid sequences that are a functional DNA sequence unit capable of effecting expression of the sequences to which they are ligated. The term “promoter” typically refers to a nucleic acid control sequence located upstream from the transcriptional start of a gene, or is operably linked to a coding sequence, and when possibly placed in the appropriate inducing conditions, is sufficient to promote transcription of said coding sequence via recognition of its sequence and binding of RNA polymerase and other proteins. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences. The term “regulatory element” also encompasses a synthetic fusion molecule or derivative that confers, activates or enhances expression of a nucleic acid molecule in a cell, tissue or organ.

The term “inducible promoter” as used herein refers to a promoter that can be switched ‘on’ or ‘off’ (thereby regulating gene transcription) in response to external stimuli such as, but not limited to, temperature, pH, certain nutrients, specific cellular signals, et cetera. It is used to distinguish between a “constitutive promoter”, by which a promoter is meant that is continuously switched ‘on’, i.e. from which gene transcription is constitutively active.

“Nucleotide sequence”, “DNA sequence”, “DNA element(s)”, or “nucleic acid molecule(s)” as used herein refers to a polymeric form of nucleotides of any length, either ribonucleotides or deoxyribonucleotides. This term refers only to the primary structure of the molecule. Thus, this term includes double- and single-stranded DNA, and RNA. It also includes known types of modifications, for example, methylation, “caps” substitution of one or more of the naturally occurring nucleotides with an analog.

“Coding sequence” is a nucleotide sequence, which is transcribed into mRNA and/or translated into a polypeptide when placed under the control of appropriate regulatory sequences. The boundaries of the coding sequence are determined by a translation start codon at the 5′-terminus and a translation stop codon at the 3′-terminus. A “coding sequence” can include, but is not limited to mRNA, cDNA, recombinant nucleotide sequences or genomic DNA, while introns may be present as well under certain circumstances. “Orthologues” are genes from different organisms that have originated through speciation, and are also derived from a common ancestral gene.

The term “vector”, as used herein, includes any vector known to the skilled person, including plasmid vectors, cosmid vectors, phage vectors, such as lambda phage, viral vectors, such as adenoviral, AAV or baculoviral vectors, or artificial chromosome vectors such as bacterial artificial chromosomes (BAC), yeast artificial chromosomes (YAC), or P1 artificial chromosomes (PAC). Said vectors include expression as well as cloning vectors. Expression vectors comprise plasmids as well as viral vectors and generally contain a desired coding sequence and appropriate DNA sequences necessary for the expression of the operably linked coding sequence in a particular host organism (e.g., bacteria, yeast, plant, insect, or mammal) or in in vitro expression systems. Cloning vectors are generally used to engineer and amplify a certain desired DNA fragment and may lack functional sequences needed for expression of the desired DNA fragments.

As used herein, “terpenoids” or otherwise “isoprenoids” refer to the large and diverse class of naturally-occurring organic chemicals of terpenes and can be found in all classes of living organisms. Terpenoids are molecules derived from a five-carbon isoprene unit that are assembled and modified in different ways and have diverse activities. Their structures are given by terpenoid biosynthesis enzymes. Plant terpenoids are used extensively for their aromatic qualities and contribute to e.g. the scent of eucalyptus, the flavours of cinnamon, clover and ginger, and the color of yellow flowers. They play a role in traditional herbal remedies and may have antibacterial, antineoplastic, and other pharmaceutical functions. Well-known terpenoids include citral, menthol, camphor, salvinorin A and cannabinoids and are also used to flavour and/or scent a variety of commercial products. The steroids and sterols in animals are biologically produced from terpenoid precursors. They also include pharmaceuticals e.g. taxol, artemisinin, vinblastine and vincristine. Terpenoids are classified with reference to the number of isoprene units that comprise the particular terpenoid. For example, a monoterpenoid comprises two isoprene units; a sesquiterpenoid comprises three isoprene units, a diterpenoid four isoprene units, and a triterpenoid six isoprene units. Polyterpenoids comprise multiple isoprene units. The synthesis of terpenoids involves a large number of enzymes with different activities. For example isoprene units are synthesized from monosaturated isoprene units by prenyltransferases into multiples of 2, 3 or 4 isoprene units. These molecules serve as substrates for terpene synthase enzymes, also called terpene cyclase. Plant terpene synthases are known in the art. “Triterpenes”, or “functionalized triterpenes” also called “triterpenoids”, all used interchangeably hereafter, consist of six isoprene units so these are composed of three terpene units with the molecular formula C₃₀H₄₈. Animals, plants and fungi all create triterpenes, with arguably the most important example being squalene as it forms the basis of almost all steroids.

A particular class of terpenoids are the saponins. The term “saponins” as used herein are a group of bio-active compounds that consist of an isoprenoidal aglycon, designated “genin” or “sapogenin”, covalently linked via a glycosidic bond to one or more sugar moieties. This combination of polar and non-polar structural elements in their molecules explains their soap-like behavior in aqueous solutions. Most known saponins are plant-derived secondary metabolites, though several saponins are also found in marine animals such as sea cucumbers and starfish. In plants, saponins are generally considered to be part of defense systems due to anti-microbial, fungicidal, allelopathic, insecticidal and moluscicidal, etc. activities. Typically, saponins reside inside the vacuoles of plant cells. Extensive reviews on molecular activities, biosynthesis, evolution, classification, and occurrence of saponins are given by e.g. Augustin et al. 2011, Phytochemistry 72:435-57, and Vincken et al. 2007, Phytochemistry 68:275-97. Thus, the term “sapogenin”, as used herein, refers to an aglycon, or non-saccharide, moiety of the family of natural products known as saponins. The commonly used nomenclature for saponins distinguishes between triterpenoid saponins (also: triterpene saponins) and steroidal saponins, which is based on the structure and biochemical background of their aglycons. Both sapogenin types are thought to derive from 2,3-oxidosqualene, a central metabolite in sterol biosynthesis. In phytosterol anabolism, 2,3-oxidosqualene is mainly cyclized into cycloartenol. Triterpenoid sapogenins branch off the phytosterol pathway by alternative cyclization of 2,3-oxidosqualene, while steroidal sapogenins are thought to derive from intermediates in the phytosterol pathway downstream of cycloartenol formation. A more detailed classification of saponins based on sapogenin structure with 11 main classes and 16 subclasses has been proposed by Vincken et al. 2007, Phytochemistry 68:275-97; particularly from page 276 to page 283). In particular, saponins may be selected from the group comprising dammarane type saponins, tirucallane type saponins, lupane type saponins, oleanane type saponins, taraxasterane type saponins, ursane type saponins, hopane type saponins, cucurbitane type saponins, cycloartane type saponins, lanostane type saponins, steroid type saponins. The aglycon backbones, the sapogenins, can be similarly classified and may be selected from the group comprising dammarane type sapogenins, tirucallane type sapogenins, lupane type sapogenins, oleanane type sapogenins, taraxasterane type sapogenins, ursane type sapogenins, hopane type sapogenins, cucurbitane type sapogenins, cycloartane type sapogenins, lanostane type sapogenins, steroid type sapogenins. A well-known example of triterpenoid saponins includes ginsenoside found in ginseng. A well-known example of steroid saponins, also referred to as glycoalkaloids, includes solanine found in potato and tomato. Triterpenoid sapogenins typically have a tetracyclic or pentacyclic skeleton. The sapogenin building blocks themselves may have multiple modifications, e.g. small functional groups, including hydroxyl, keto, aldehyde, and carboxyl moieties, of precursor sapogenin backbones such as β-amyrin, lupeol, and dammarenediol. It is to be understood that the triterpenoid sapogenins, as used herein, also encompass new-to-nature triterpenoid compounds which are structurally related to the naturally occurring triterpenoid sapogenins. These new-to-nature triterpenoid sapogenins may be currently unextractable compounds by making use of existing extraction procedures or may be novel compounds that can be obtained after genetic engineering of the synthesizing eukaryotic host cell.

The term “endogenous” as used herein, refers to substances (e.g. genes) originating from within an organism, tissue, or cell. Analogously, “exogenous” as used herein is any material originated outside of an organism, tissue, or cell, but that is present (and typically can become active) in that organism, tissue, or cell.

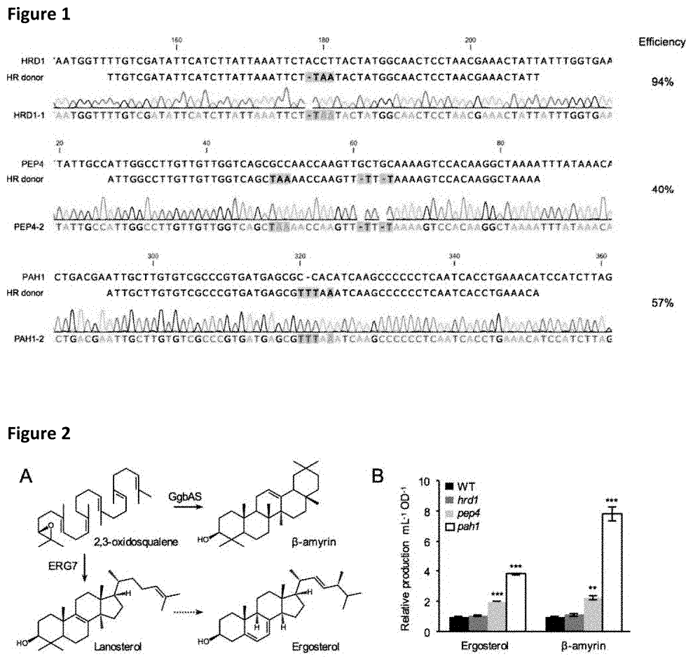

The term “phosphatidic acid phosphatase” or “PAP” is used herein to designate an enzyme catalyzing the dephosphorylation of phosphatidic acid (PA) (EC 3.1.3.4), thereby yielding diacylglycerol (DAG) and phosphate (Pi). Most particularly, PAP is specific for PA and requires Mg²⁺ for activity, to distinguish from lipid phosphate phosphatase, also designated as PAP2, which is not specific for phosphatidic acid and does not require Mg²⁺ for activity (although it helps in reaching maximal activity) (Carman and Han, 2006). The term “PAH1” as used herein refers to the yeast PAP enzyme and the encoding gene (Gene ID: 855201 in Saccharomyces cerevisiae; gene and protein sequences of the Yarrowia lipolytica and Pichia pastoris PAH1 are shown in FIGS. 1 and 2 of WO2011157761, including an alignment with the Saccharomyces cerevisiae PAH1 protein), sometimes also indicated as SMP2 (Santos-Rosa, H. et al., EMBO J., 2005; Han, G. S. et al., J Biol Chem., 2006).

A “PAH1 homolog” as used throughout the application refers to genes and proteins in species other than yeast homologous to PAH1 and having PAP activity. Homology is expressed as percentage sequence identity (for nucleic acids and amino acids) and/or as percentage sequence similarity (for amino acids). Preferably, homologous sequences show at least 50%, at least 60%, at least 70%, at least 80%, at least 90%, at least 95% or at least 99% sequence identity at nucleic acid level or sequence identity or similarity at amino acid level. Algorithms to determine sequence identity or similarity by sequence alignment are known to the person skilled in the art and include for instance the BLAST program. Alternatively, homologs can be identified using the HomoloGene database (NCBI) or other specialized databases such as for instance HOGENOM or HOMOLENS (Penel, L. et al., BMC Bioinformatics., 2009). Examples of PAH1 homologs include, but are not limited to, lipins in mammalians and some other vertebrates (encoded by Lpin1, Lpin2, and Lpin3; Gene ID: 23175, 9663, and 64900 in humans and 14245, 64898 and 64899 in mice, respectively), nedl in Schizosaccharomyces (GeneID: 2542274), CG8709 in Drosophila (GeneID: 35790), AgaP_AGAP007636 in Anopheles (GeneID: 1269590), and AT3G09560 (GeneID: 820113) and AT5G42870 (GeneID: 834298) in Arabidopsis thaliana. A particularly envisaged PAH1 homolog is lipin-1. Typical of these PAH1 homologs is that they possess a NLIP domain with a conserved glycine residue at the N-terminus and a HAD-like domain with conserved aspartate residues in the catalytic sequence DIDGT (SEQ ID NO: 11) (Péterfy, M. et al., Nat Genet., 2001; Han, G. S. et al., J Biol Chem., 2007; Carman and Han, Biol Chem., 2009).

The term “diacylglycerol kinase”, “DAGK” or “DGK” as used herein refers to an enzyme catalyzing the reverse reaction as a phosphatidic acid phosphatase, i.e. the phosphorylation of DAG to obtain phosphatidic acid (EC 2.7.1.107 for the ATP-dependent DGK; in yeast, the enzyme is CTP-dependent (Han, G. S. et al., J Biol Chem., 2008a and 2008b) and EC 2.7.1.n5 has been proposed as nomenclature in the Uniprot database).

The term “DGK1” as used herein refers to the yeast DGK enzyme and the encoding gene (GeneID: 854488 in Saccharomyces cerevisiae; Gene ID: 8199357 in Pichia Pastoris and Gene ID: 2909033 for Yarrowia lipolytica). The gene and protein sequences of these DGK1s are also shown in FIG. 6 of WO2011157761, sometimes also indicated as HSD1.

A “DGK1 homolog” as used throughout the application refers to genes and proteins in species other than yeast homologous to DGK1 and having diacylglycerol kinase activity. Homology is as detailed above. DGK1 homologs are found throughout the eukaryotes, from yeast over plants (Katagiri, T. et al., Plant Mol Biol., 1996; Vaultier, M. N. et al., FEBS Lett., 2008) to C. elegans (Jose and Koelle, J Biol Chem., 2005) and mammalian cells (Sakane, F. et al., Biochim Biophys Acta., 2007). Typically, DGK1 in yeast uses CTP as the phosphate donor in its reaction (Han, G. S. et al., J Biol Chem., 2008b) while DGK1 homologs in e.g. mammalian cells use ATP instead of CTP (Sakane, F. et al., Biochim Biophys Acta., 2007).

“OPI1” is a transcriptional repressor in yeast (Gene ID: 856366 for Saccharomyces cerevisiae; Gene ID: 2909741 for Yarrowia lipolytica). It is a negative regulator of the transcriptional complex INO2-INO4 in response to phospholipid precursor availability. When precursors become limiting, OPI1 is retained at the endoplasmic reticulum (ER) and INO2-INO4 activates INO1 and other genes required for phospholipid biosynthesis, whereas abundant precursor availability results in targeting of OPI1 to the nucleus to repress transcription of these genes. OPI1 binds directly to phosphatidic acid, which is required for ER targeting and may act as sensing mechanism for precursor availability, as phosphatidic acid becomes rapidly depleted upon phospholipid biosynthesis.

“INO2” also known as “INOsitol requiring2” is a component of the heteromeric Ino2p/Ino4p basic helix-loop-helix transcription activator that binds inositol/choline-responsive elements required for depression of phospholipid biosynthetic genes in response to inositol depletion (Gene ID: 851701 for Saccharomyces cerevisiae).

“INO4” is the other component of said heteromeric Ino2p/Ino4p basic helix-loop-helix transcription activator (Gene ID: 854042 for Saccharomyces cerevisiae).

“GIS1” also known as “Glg1-2 Suppressor” in yeast is a histone demethylase and transcription factor (SGD ID: S000002503).

“RPH1” also known as “Regulator of PHR1”, is a JmjC domain-containing histone demethylase (SGD ID: S000000971).

“NEM1” also known as “Nuclear Envelope Morphology1” is the catalytic subunit of the Nem1p-Spo7p phosphatase holoenzyme (SGD ID: S000001046).

“SPO7” also known as “SPOrulation7” or “SPOrulation specific protein7” is the regulatory subunit of Nem1p-Spo7p phosphatase holoenzyme (SGD ID: S000000007).

It is an object of the invention to provide cells or cellular systems (cultures, organisms) that can produce high amounts of terpenoids, particularly terpenoids that depend on intracellular membrane related enzymes for their biosynthesis. Also, methods are provided that use such cells or cellular systems to produce higher amounts of such terpenoids than is feasible with existing methods.

According to a first aspect, recombinant eukaryotic cells are provided wherein the proliferation of the intracellular membrane is increased in comparison with a control cell. This is equivalent as saying that a recombinant eukaryotic cell is provided with increased intracellular membrane proliferation or an increased intracellular membrane system compared to a control cell. Said recombinant eukaryotic cells further comprise at least one chimeric gene construct comprising a promoter active in said recombinant eukaryotic cells operably fused to a nucleic acid sequence which encodes a terpenoid biosynthesis enzyme. In one embodiment, a recombinant eukaryotic cell is provided with an expanded endoplasmic reticulum compared to a control cell, wherein said recombinant eukaryotic cell further comprising at least one chimeric gene construct comprising a promoter active in said recombinant eukaryotic cell operably fused to a nucleic acid sequence which encodes a terpenoid biosynthesis enzyme. In particular embodiments, said expanded endoplasmic reticulum is an endoplasmic reticulum that is at least 10%, at least 20%, at least 30%, at least 40%, at least 50%, at least 75%, at least 100% more expanded compared to that of a control cell. Analogously, the recombinant eukaryotic cell that is provided with increased intracellular membrane proliferation compared to a control cell has an intracellular membrane proliferation of at least 10%, at least 20%, at least 30%, at least 40%, at least 50%, at least 75%, at least 100% more compared to said control cell. An expansion or the proliferation of the endoplasmic reticulum (ER) can easily be assessed visually using electron microscopy or using fluorescent markers specifically labelling the ER.

In particular embodiments according to the invention, proliferation of the intracellular membrane more particularly of the ER is increased due to an inhibition of negative regulation of said proliferation. In the application it is disclosed that phosphatidic acid phosphatase (PAP) activity negatively regulates the proliferation or expansion of the intracellular membrane compartment, more particularly of the ER. Therefore, in yet other particular embodiments, increased intracellular membrane proliferation more particularly of the ER is achieved by inhibition of the expression and/or activity of an endogenous phosphatidic acid phosphatase. PAP activity or the conversion of phosphatidate to diacylglycerol is counteracted by diacylglycerol kinase. Therefore in yet another embodiment, increased intracellular membrane proliferation or increased ER proliferation is achieved by overexpressing a diacylglycerol kinase.

PAP activity in yeast is performed by PAH1. PAH1 expression is controlled by two regulatory complexes, the Ino2p/Ino4p/Opi1p regulatory circuit and the transcription factors Gis1p and Rph1p which bind to different positions on the PAH1 promoter and as such induce gene expression. PAH1 activity is also known to be controlled posttranslationally by phosphorylation. Indeed, the Nem1/Spo7 phosphatase complex activates PAH1 by dephosphorylating the enzyme. Reducing the expression and/or activity of the regulatory complexes is disclosed herein to reduce PAP activity and thus to increase the intracellular membrane system more particularly the ER. Indeed, in Example 10 it is demonstrated that inhibition of Opi1 induces the production of terpenoids. Therefore, in other embodiments, a recombinant eukaryotic cell is provided with increased intracellular membrane proliferation compared to a control cell, wherein said recombinant eukaryotic cell further comprising at least one chimeric gene construct comprising a promoter active in said recombinant eukaryotic cell operably fused to a nucleic acid sequence which encodes a terpenoid biosynthesis enzyme, wherein said increased intracellular membrane proliferation more particularly of the ER is achieved by inhibition of the expression and/or activity of a PAH1 regulator selected from the list consisting of Opi1, Ino2, Ino4, Gis1, Rph1, Nem1 and Spo7. In more particular embodiments, said increased intracellular membrane proliferation more particularly of the ER is achieved by inhibition of the expression and/or activity of Opi1, Ino2, Ino4, Gis1 or Rph1. In other particular embodiments, said increased intracellular membrane proliferation more particularly of the ER is achieved by inhibition of the expression and/or activity of Nem1 or Spo7.

In Example 11, it is disclosed that PAH1 activity can also be controlled in a pharmacological manner, using propranolol, sphingosine, sphinganine, rutin, kaempferol, N-ethylmaleimide or bromoenol lactone. Therefore, a recombinant eukaryotic cell is provided with increased intracellular membrane proliferation compared to a control cell, wherein said recombinant eukaryotic cell further comprising at least one chimeric gene construct comprising a promoter active in said recombinant eukaryotic cell operably fused to a nucleic acid sequence which encodes a terpenoid biosynthesis enzyme, wherein said increased intracellular membrane proliferation more particularly of the ER is achieved by applying to said eukaryotic cell a compound selected from the list consisting of propranolol, sphingosine, sphinganine, rutin, kaempferol, N-ethylmaleimide and bromoenol lactone.

Propranolol (C₁₆H₂₁NO₂; CAS 525-66-6; PubChem CID 4946) is a well-known drug of the beta blocker type that is commercially available. As a beta-adrenergic receptor antagonist it is used to treat high blood pressure and a number of irregular heart rate types. Here it is disclosed that propranolol, propranolol hydrochloride and variants thereof can also be used to increase the production of terpenoids in recombinant yeast cells comprising a chimeric gene construct comprising a promoter active in said cell operably fused to a nucleic acid sequence which encodes a terpenoid biosynthesis enzyme. Propranolol is defined by the structural formula:

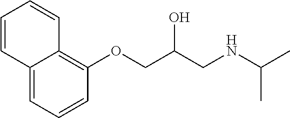 [SMILES: CC(C)NCC(O)COc1cccc2ccccc12]

In a most particular embodiment, the use of propranolol, propranolol hydrochloride or variants thereof is provided to increase the production of a terpenoid in a eukaryotic cell. In even more particular embodiments, said eukaryotic cell is a plant cell or is a recombinant eukaryotic cell comprising a chimeric gene construct comprising a promoter active in said recombinant eukaryotic cell operably fused to a nucleic acid sequence which encodes a terpenoid biosynthesis enzyme.

N-Ethylmaleimide (NEM) (C₆H₇NO₂; CAS 128-53-0; PubChem CID 4362) is an organic compound that is derived from maleic acid. It contains the imide functional group, but more importantly it is an alkene that is reactive toward thiols and is commonly used to modify cysteine residues in proteins and peptides. It is also known as 1-ethylpyrrole-2,5-dione or ethylmaleimide and has the following structural formula:

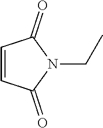

In a most particular embodiments, the use of N-ethylmaleimide or variants thereof is provided to increase the production of a terpenoid in a eukaryotic cell. In even more particular embodiments, said eukaryotic cell is a plant cell or is a recombinant eukaryotic cell comprising a chimeric gene construct comprising a promoter active in said recombinant eukaryotic cell operably fused to a nucleic acid sequence which encodes a terpenoid biosynthesis enzyme.

Bromoenol lactone (BEL) (C16H13BrO2; CAS 478288-90-3) is an inhibitor of calcium-independent phospholipase γ (iPLA2γ) (Tsuchida et al 2015 Mediators Inflamm 605727). The calcium-independent phospholipases (iPLA2) are a PLA2 subfamily closely associated with the release of arachidonic acid in response to physiologic stimuli. BEL has the following structural formula:

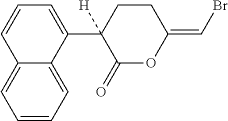 [SMILES: O=C1O/C(=C/Br)CC[C@@H]1c1cccc2ccccc12]

In a most particular embodiments, the use of bromoenol lactone or variants thereof is provided to increase the production of a terpenoid in a eukaryotic cell. In even more particular embodiments, said eukaryotic cell is a plant cell or is a recombinant eukaryotic cell comprising a chimeric gene construct comprising a promoter active in said recombinant eukaryotic cell operably fused to a nucleic acid sequence which encodes a terpenoid biosynthesis enzyme.

Kaempferol (C₁₅H₁₀O₆; CAS 520-18-3; PubChem CID 5280863) also known as 3,5,7-Trihydroxy-2-(4-hydroxyphenyl)-4H-chromen-4-one, kaempherol, robigenin, pelargidenolon, rhamnolutein, rhamnolutin, populnetin, trifolitin, kempferol or swartziol is a natural flavonol, a type of flavonoid, found in a variety of plants and plant-derived foods. Kaempferol acts as an antioxidant by reducing oxidative stress. Kaempferol has the following structural formula:

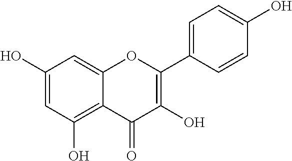 [SMILES: O=c1c(O)c(-c2ccc(O)cc2)oc2cc(O)cc(O)c12]

In a most particular embodiments, the use of kaempferol or variants thereof is provided to increase the production of a terpenoid in a eukaryotic cell. In even more particular embodiments, said eukaryotic cell is a plant cell or is a recombinant eukaryotic cell comprising a chimeric gene construct comprising a promoter active in said recombinant eukaryotic cell operably fused to a nucleic acid sequence which encodes a terpenoid biosynthesis enzyme.

Rutin (C₂₇H₃₀O₁₆; CAS 153-18-4; PubChem CIB 5280805) also known as rutoside, phytomelin, sophorin, birutan, eldrin, birutan forte, rutin trihydrate, globularicitrin, violaquercitrin, quercetin-3-O-rutinoside, quercetin rutinoside or 2-(3,4-dihydroxyphenyl)-5,7-dihydroxy-3-[α-L-rhamnopyranosyl-(1→6)-β-D-glucopyranosyloxy]-4H-chromen-4-one, is the glycoside combining the flavonol quercetin and the disaccharide rutinose (α-L-rhamnopyranosyl-(1→6)-β-D-glucopyranose). Rutin is a citrus flavonoid found in a wide variety of plants including citrus fruit with the following structural formula:

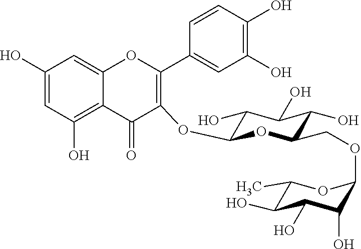

In a most particular embodiments, the use of rutin or variants thereof is provided to increase the production of a terpenoid in a eukaryotic cell. In even more particular embodiments, said eukaryotic cell is a plant cell or is a recombinant eukaryotic cell comprising a chimeric gene construct comprising a promoter active in said recombinant eukaryotic cell operably fused to a nucleic acid sequence which encodes a terpenoid biosynthesis enzyme.

Sphinganine (C₁₈H₃₉NO₂; CAS 764-22-7; PubChem CID 4094) also known as dihydrosphingosine or 2-amino-1,3-dihydroxyoctadecane is a blocker postlysosomal cholesterol transport by inhibition of low-density lipoprotein-induced esterification of cholesterol. Sphinganine causes unesterified cholesterol to accumulate in perinuclear vesicles. It has been suggested the possibility that endogenous sphinganine may inhibit cholesterol transport in Niemann-Pick Type C (NPC) disease (Roff et al 1991 Dev Neurosci 13:315-319). Here, it is disclosed that sphinganine (structural formula below) can be used to increase the production of a terpenoid. Sphinganine has the following formula:

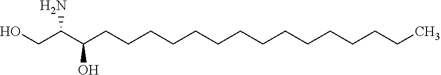 [SMILES: CCCCCCCCCCCCCCC[C@@H](O)[C@@H](N)CO]

In a most particular embodiments, the use of sphinganine or variants thereof is provided to increase the production of a terpenoid in a eukaryotic cell. In even more particular embodiments, said eukaryotic cell is a plant cell or is a recombinant eukaryotic cell comprising a chimeric gene construct comprising a promoter active in said recombinant eukaryotic cell operably fused to a nucleic acid sequence which encodes a terpenoid biosynthesis enzyme.

Sphingosine (C₁₈H₃₇NO₂; CAS 123-78-4; PubChem CID 5280335) also known as 2-amino-4-octadecene-1,3-diol is an 18-carbon amino alcohol with an unsaturated hydrocarbon chain, which forms a primary part of sphingolipids, a class of cell membrane lipids that include sphingomyelin, an important phospholipid. Sphingosine has the following formula:

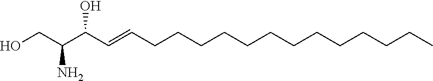 [SMILES: CCCCCCCCCCCCC/C=C/[C@@H](O)[C@@H](N)CO]

In a most particular embodiments, the use of sphinganine or variants thereof is provided to increase the production of a terpenoid in a eukaryotic cell. In even more particular embodiments, said eukaryotic cell is a plant cell or is a recombinant eukaryotic cell comprising a chimeric gene construct comprising a promoter active in said recombinant eukaryotic cell operably fused to a nucleic acid sequence which encodes a terpenoid biosynthesis enzyme.

Terpenoids that can be produced in the recombinant eukaryotic cells and using the methods according to the invention are typically selected from hemiterpenoids, monoterpenoids, sesquiterpenoids, diterpenoids, sesterpenoids, triterpenoids, tetraterpenoids, polyterpenoids or glycosides thereof. In one embodiment, the terpenoid is a triterpenoid, a sesquiterpenoid or a saponin.

In a specific embodiment, the terpenoid is beta-amyrin. In another specific embodiment, the terpenoid is Glycyrrhetinic acid. In yet another specific embodiment, the terpenoid is artemisinic acid. In another specific embodiment, the terpenoid is thalianol. In another specific embodiment, the terpenoid is Lupeol. In yet another specific embodiment, the terpenoid is Betulinic acid. In another specific embodiment, the terpenoid is alpha-amyrin. In a specific embodiment, the terpenoid is Protopanaxatriol. In another specific embodiment, the terpenoid is 11-oxo-cucurbitadienol. In yet another specific embodiment, the terpenoid is Costunolide. In a specific embodiment, the terpenoid is (+)-nootkatone. In another specific embodiment, the terpenoid is α-farnesene. In yet another specific embodiment, the terpenoid is taxadiene. In other specific embodiments, the terpenoid is ergosterol, erythrodiol, oleandic aldehyde, oleandic acid, botulin, betulinic aldehyde, hederagenin, 2-OH-oleandic acid, gypsogenic acid, bayogenin, medicagenic acid, 24-hydroxy-beta-amyrin, 24-carboxy-beta-amyrin, dihydrolupeol, Glc-bayogenin, Glc-hederagenin, Glc-gypsogenic acid or Glc-medicagenic acid.

According to the present disclosure, the skilled person can select a terpenoid to be produced in the recombinant eukaryotic cells according to the invention. In general, the production of every terpenoid can be envisaged as long as the biosynthesis genes for said terpenoid are present in or are provided to the eukaryotic or recombinant eukaryotic cell. Typically, when a terpenoid is produced in a recombinant eukaryotic cell according to the invention, the production yield of said terpenoid increases by at least 10, 20, 30, 40, 50, 60, 70, 80, 90, 100, 200, 300, 400, 500, 600, 700, 800, 900, 1000 or even 2000% compared to a terpenoid produced in a control cell.

In particular embodiments, the recombinant eukaryotic cells according to the invention are yeast cells and in even more particular embodiments cells of the species Saccharomyces cerevisiae. The cells will further typically contain a nucleic acid sequence encoding a terpenoid biosynthesis enzyme to be expressed in said cells. Most particularly, the nucleic acid sequence is an exogenous sequence or an endogenous sequence under control of an exogenous promoter. In most particular embodiments, said nucleic acid sequence is a plant nucleic acid sequence. In even more particular embodiments, said nucleic acid sequence encodes a plant P450 enzyme.

Accordingly, methods of enhancing terpenoid production in such recombinant eukaryotic cells are provided. These methods entail that a recombinant eukaryotic cell with an increased ER proliferation is provided, wherein the cell comprises a nucleic acid sequence encoding the terpenoid biosynthesis enzyme of interest and the cell is maintained in conditions suitable for expressing the triterpenoid biosynthesis enzyme. The produced terpenoid may further optionally be isolated and/or purified. The nature of the eukaryotic cells, both as such and as used in the methods provided herein, can be very varied, since all eukaryotic cells have an intracellular membrane and it is through expansion of this membrane that terpenoid production is increased. Also, phosphatidic acid phosphatases and diacylglycerol kinases occur in all kinds of eukaryotic cells, and it has been shown that their function is evolutionarily conserved from unicellular eukaryotes to mammals (Grimsey, N. et al., Biol Chem., 2008). In this regard, it should be stressed that the technical effect of PAP inhibition is identical to that of increasing DGK activity, since the enzymes catalyze opposite directions of the same reaction. It is particularly envisaged that the eukaryotic cells used are eukaryotic cells that are normally used as expression systems, to take further advantage of optimized terpenoid production. Examples of eukaryotic cells that are used for protein production include, but are not limited to, yeast cells (e.g. Pichia, Hansenula, Yarrowia), insect cells (e.g. SF-9, SF-21, and High-Five cells), mammalian cells (e.g. Hek293, COS, CHO cells), plant cell cultures (e.g. Nicotiana tabacum, Oryza sativa, soy bean or tomato cultures, see for instance Hellwig, S. et al., Nat Biotechnol., 2004; Huang, T. K. et al., Biochemical Engineering Journal, 2009), or even whole plants. The cells may thus be provided as such, as a eukaryotic cell culture, or even as an organism (i.e. a non-human organism). According to particular embodiments, however, the organism is not a mouse, or not even a mammal.

It is particularly envisaged that the eukaryotic cells are yeast cells, as these are very amenable to protein production and are robust expression systems. According to particular embodiments, the yeast cells are from the genus Saccharomyces. In even more particular embodiments the yeast cells are from the species Saccharomyces cerevisiae. According to even more particular embodiments, the yeast cells are methylotrophic yeast cells, such as species of the genus Hansenula (e.g. Hansenula polymorpha), species of the genus Candida (e.g. Candida boidinii) or most particularly species of the genus Pichia, such as Pichia pastoris. According to alternative embodiments, the yeast cells are of the genus Yarrowia, most particularly of the species Yarrowia lipolytica. Typically, the terpenoid that is produced in a yeast cell will be isolated (or possibly secreted) from the cell.

According to alternative particular embodiments, the eukaryotic cells are plant cells, particularly plant cell cultures. It should be clear to the skilled person that even whole plants can be used. Thus, in one embodiment according to the invention the whole plant is used for protein or metabolite production. In one particular embodiment, the whole plant used for protein or metabolite production is Nicotiana benthamiana. According to yet further alternative embodiments, the eukaryotic cells are mammalian cells, most particularly Hek293 cells, such as Hek293S cells.

To make a cell deficient in expression and/or activity of an endogenous phosphatidic acid phosphatase, several strategies can be used, and the nature of the strategy is not vital to the invention, as long as it results in diminishing PAP activity to the extent that the intracellular membrane is expanded in the cell. Cells can be made deficient for PAP at the genetic level, e.g. by deleting, mutating, replacing or otherwise disrupting the (endogenous) gene encoding PAP. Alternatively, one can interfere with transcription from the PAP gene, or remove or inhibit the transcribed (nucleic acid, mRNA) or translated (amino acid, protein) gene products. This may for instance be achieved through siRNA inhibition of the PAP mRNA. Also morpholinos, miRNAs, shRNA, LNA, small molecule inhibition or similar technologies may be used, as the skilled person will be aware of. The PAP protein can for instance be inhibited using inhibitory antibodies, antibody fragments, scFv, Fc or nanobodies, small molecules or peptides.

Another way in which genes such as PAH1 can be knocked out is by the use endonuclease technology which includes but is not limited to the use of zinc finger nucleases, TALEN, Crispr/Cas, meganucleases. Zinc-finger nucleases (ZFNs) are artificial restriction enzymes generated by fusing a zinc finger DNA-binding domain to a DNA cleavage domain. Zinc finger domains can be engineered to target desired DNA sequences, which enable zinc-finger nucleases to target unique sequence within a complex genome. By taking advantage of endogenous DNA repair machinery, these reagents can be used to precisely alter the genomes of higher organisms. Other technologies for genome customization that can be used to knock out genes are meganucleases and TAL effector nucleases (TALENs, Cellectis bioresearch). A TALEN® is composed of a TALE DNA binding domain for sequence-specific recognition fused to the catalytic domain of an endonuclease that introduces double strand breaks (DSB). The DNA binding domain of a TALEN® is capable of targeting with high precision a large recognition site (for instance 17 bp). Meganucleases are sequence-specific endonucleases, naturally occurring “DNA scissors”, originating from a variety of single-celled organisms such as bacteria, yeast, algae and some plant organelles. Meganucleases have long recognition sites of between 12 and 30 base pairs. The recognition site of natural meganucleases can be modified in order to target native genomic DNA sequences (such as endogenous genes). Another recent genome editing technology is the CRISPR-Cas system, which can be used to achieve RNA-guided genome engineering. CRISPR interference is a genetic technique which allows for sequence-specific control of gene expression in prokaryotic and eukaryotic cells. It is based on the bacterial immune system-derived CRISPR (clustered regularly interspaced palindromic repeats) pathway that confers resistance to foreign genetic elements such as those present within plasmids and phages providing a form of acquired immunity. A simple version of the CRISPR/Cas system, CRISPR/Cas9, has been modified to edit genomes. By delivering the Cas9 nuclease complexed with a synthetic guide RNA (gRNA) into a cell, the cell's genome can be cut at a desired location, allowing existing genes to be removed and/or new ones added (Marraffini and Sontheimer 2010 Nat Rev Genet 11:181-190). In meantime, alternatives for the Cas9 nuclease have been identified, e.g. Cpf1 or Cas12 (Zetsche et al 2015 Cell 3:759-771). Recently, it was demonstrated that the CRISPR-Cas editing system can also be used to target RNA. It has been shown that the Class 2 type VI-A CRISPR-Cas effector C2c2 (also known as Cas13) can be programmed to cleave single stranded RNA targets carrying complementary protospacers (Abudayyet et al 2016 Science aaf5573; Abudayyet et al 2017 Nature 5:280-284). C2c2 is a single-effector endoRNase mediating ssRNA cleavage once it has been guided by a single crRNA guide toward the target RNA. This system can thus also be used to target and thus to break down LIPIN, LIPIN1, CTDNEP1 or CNEP1R1. Expression of PAH1 can also be inhibited indirectly by targeting the complex that regulates the expression of PAH1. On transcriptional level, PAH1 expression is known to be controlled by two regulatory complexes, the Ino2p/Ino4p/Opilp regulatory circuit and the transcription factors Gis1p and Rph1p which bind to different positions of the PAH1 promoter and as such induce gene expression. Indeed, it was demonstrated that loss-of-function of either of the components leads to decreased PAH1 expression (Pascual, F. et al., Journal of Biological Chemistry, 2013) while in Example 10 it is demonstrated that reduced INO2 expression leads to increase terpenoid production. The person skilled in the art is thus fully taught about the alternatives to increase the intracellular membrane compartment through reduction of PAH1 expression or of positive regulators of PAH1 expression in order to increase the production of terpenoids.

Interestingly, PAP activity may also be inhibited without directly interfering with PAP expression products. For instance, in yeast it has been shown that the loss of the dephosphorylated form of the yeast PAP enzyme PAH1 by deletion of Nem1 and/or Spo7, which form a complex that dephosphorylates PAH1, results in the same phenotype as deletion of PAH1 (Siniossoglou, S. et al, EMBO J., 1998; Santos-Rosa, H. et al., EMBO J., 2005). Likewise, increased phosphorylation of Lipin 1 and 2 inhibits their PA phosphatase activity (Grimsey, N. et al., Biol Chem., 2008). Thus, a cell may be made deficient in PAP activity by increasing PAP phosphorylation (or blocking PAP dephosphorylation). As will be clear to those of skill in the art, deficiency of PAP expression and/or activity may both be constitutive (e.g. genetic deletion) or inducible (e.g. small molecule inhibition). In particular embodiments, the endogenous phosphatidic acid phosphatase is PAH1 or a homolog thereof. The skilled person should be aware of the large number of reports discussing several opportunities to interfere with PAH1 expression and activity.

The skilled person can use means and methods disclosed herein in order to increase intracellular membrane proliferation or increase the intracellular membrane compartment of the recombinant eukaryotic cells according to the invention to achieve increased terpenoid production.

For instance and non-limiting, Pascual, F. et al., J Biol Chem., 2013 describes the interference via the Ino2p/Ino4p/Opilp regulatory circuit and transcription factors Gis1p and Rph1p (see also Ruijter, J. C. de et al., Microb Cell Fact., 2016) while an approach based on TORC1 regulating Pah1 phosphatidate phosphatase activity via the Nem1/Spo7 protein phosphatase complex is disclosed in Dubots, E. et al., PLoS One., 2014. In a particular embodiment according to the invention, increased intracellular membrane proliferation is achieved by knockout of OP11. In yet another particular embodiment according to the invention, increased intracellular membrane proliferation is achieved by knockout of Spo7.

It should be clear to the skilled person based on the disclosure presented herein that different methods to increase intracellular membrane proliferation can be combined with each other and are also within the scope of the invention as presented. As a non-limiting example illustrating this approach a knockout of both OPI1 and SPO7 can be combined.

Also within the scope of the present invention is the combination of methods to achieve increased intracellular membrane proliferation with methods to increase protein stability. Methods and knockouts in particular to increase protein stability are known to the skilled person. Illustrating and non-limiting, a knockout of PEP4 can be combined with methods to increase protein stability. PEP4 as referred to herein encodes aspartyl protease proteinase A and is involved in the maturation of several vacuolar peptidases in yeast (Parr, C. L. et al., Yeast, 2007). PEP4 deficiency was repeatedly reported as beneficial for the stability of heterologously expressed proteins in yeast (Liao, M. et al., Mol. Pharmacol., 2005; Oka, T. et al., J. Biol. Chem., 2007). Thus, the present disclosure also envisages recombinant eukaryotic cells deficient in PEP4 for the increased production of terpenoids.

The eukaryotic cells provided herein may, either solely or in addition to PAP deficiency, overexpress a diacylglycerol kinase. This may be endogenous DGK that is overexpressed, for instance by means of an exogenous promoter. The exogenous promoter may be constitutive or inducible, but typically will be a stronger promoter than the endogenous promoter, to ensure overexpression of DGK. Alternatively, an exogenous diacylglycerol kinase is overexpressed, i.e. the eukaryotic cell is genetically engineered so as to express a DGK that it does not normally express. The exogenous DGK may for instance also be a non-naturally occurring DGK, such as for instance a functional fragment of a diacylglycerol kinase. A fragment is considered functional if it retains the capability to catalyze the phosphorylation reaction of DAG to obtain phosphatidic acid. As for endogenous DGK, the exogenous DGK may also be under control of a constitutive or inducible promoter. The nature of the promoter is not vital to the invention and will typically depend on the expression system (cell type) used and/or on the amount of protein that is needed or feasible. According to particular embodiments, the diacylglycerol kinase that is overexpressed is DGK1 or a homolog thereof.

Particularly envisaged are cells that combine a deficiency in endogenous PAP with overexpression of a DGK, for even higher production of proteins, although the effect is not necessarily additive. Note that the cells described herein that are characterized by significant intracellular membrane expansion (by having a PAP deficiency and/or overexpressing a DGK) may be further engineered for increased terpenoid biosynthesis enzyme expression. A non-limiting example thereof is overexpression of HAC1 (Guerfal, M. et al., Microb Cell Fact., 2010), but other modifications are also known in the art. Alternatively or additionally, the cells may be further engineered to perform eukaryotic post-translational modifications (e.g. De Pourcq, K. et al., Appl Microbiol Biotechnol., 2010).

In another aspect, the use of a eukaryotic cell is provided for the production of terpenoids, wherein said eukaryotic cell has an increased intracellular membrane compartment compared to a control cell and wherein said eukaryotic cell comprises a nucleic acid sequence encoding a terpenoid biosynthesis enzyme. In one embodiment, said eukaryotic cell is a plant cell. In another embodiment, said eukaryotic cell is a recombinant eukaryotic cell which comprises at least one chimeric gene construct comprising a promoter active in said recombinant eukaryotic cell operably linked to a nucleic acid sequence encoding a terpenoid biosynthesis enzyme.

In another aspect, the use of an inhibitor of PAH1 is provided for the production of a terpenoid in a eukaryotic cell. In one embodiment, said eukaryotic cell is a plant cell. In another embodiment, said eukaryotic cell is a recombinant eukaryotic cell which comprises at least one chimeric gene construct comprising a promoter active in said recombinant eukaryotic cell operably linked to a nucleic acid sequence encoding a terpenoid biosynthesis enzyme. In a particular embodiment, said inhibitor is selected from the list consisting of propranolol, sphingosine, sphinganine, rutin, kaempferol, N-ethylmaleimide and bromoenol lactone. In another particular embodiment, said terpenoid is produced at a higher level or in a bigger amount upon using the PAH1 inhibitor.

In yet another aspect, methods are provided for the production of a terpenoid in a recombinant eukaryotic cell, comprising providing a recombinant eukaryotic cell wherein the intracellular membrane proliferation is increased or wherein the intracellular membrane compartment is expanded in comparison with a control cell and introducing in said recombinant eukaryotic cell at least one chimeric gene construct comprising a promoter active in said recombinant eukaryotic cell operably fused to a nucleic acid sequence encoding a terpenoid biosynthesis enzyme in conditions suitable for producing the terpenoid. In one embodiment, the proliferation of the intracellular membrane compartment is due to inhibition of the negative regulation of intracellular membrane proliferation. In another embodiment, the expression and/or activity of an endogenous phosphatidic acid phosphatase is inhibited, and/or a diacylglycerol kinase is overexpressed or has an increased activity of an endogenous diacylglycerol kinase. In a particular embodiment, said endogenous phosphatidic acid phosphatase is PAH1 and/or said diacylglycerol kinase is DGK1.

In another aspect, a method is provided for the production or increased production of a terpenoid in a plant cell, wherein said plant cell comprises at least one nucleic acid sequence encoding a terpenoid biosynthesis enzyme, comprising treating said plant cell with an effective amount of a PAP inhibitor. In one embodiment, said PAP inhibitor is a PAH1 inhibitor. In a more particular embodiment, said PAP inhibitor or PAH1 inhibitor is selected from the list consisting of propranolol, sphingosine, sphinganine, rutin, kaempferol, N-ethylmaleimide and bromoenol lactone. In a particular embodiment, a method is provided for the increased production of a terpenoid in a plant cell, wherein said plant cell comprises at least one nucleic acid sequence encoding a terpenoid biosynthesis enzyme, comprising treating said plant cell with an effective amount of propranolol.

The terpenoid biosynthesis enzymes that are produced in the cells described herein will typically be encoded by an exogenous nucleic acid sequence or an endogenous nucleic acid sequence under control of an exogenous promoter, i.e. the cells are engineered to express the terpenoid biosynthesis enzyme of interest. The terpenoid biosynthesis enzyme may be expressed constitutively or in an inducible way. Accordingly, the promoter may be a constitutive or inducible promoter.

According to particular embodiments, the terpenoids that are produced in the eukaryotic cells described herein are terpenoids that rely on intracellular membrane related enzymes for their biosynthesis. According to other particular embodiments, the terpenoids that are produced in the eukaryotic cells described herein are terpenoids that rely on non-intracellular membrane related enzymes for their biosynthesis. According to yet other particular embodiments, the terpenoids that are produced in the eukaryotic cells described herein are terpenoids that rely on both intracellular and non-intracellular membrane related enzymes for their biosynthesis.

According to specific embodiments, more than one, i.e. two or more different terpenoids may be produced simultaneously. The terpenoid biosynthesis enzymes may all be intracellular membrane-related, may all be non-intracellular membrane related, may be both intracellular and non-intracellular membrane related, all be secreted terpenoids or a mixture thereof. When more than one terpenoid is produced, care will typically be taken that they can be recovered easily either separately or together. In a specific embodiment, even higher production is achieved by expressing multiple copies of the terpenoid biosynthesis enzymes to be expressed, e.g. as a polyprotein.

During or after the terpenoid production in the recombinant eukaryotic cells, the terpenoid or terpenoids of interest can be recovered from the cells. Accordingly, the methods of terpenoid production may optionally also comprise the step of isolating the produced terpenoid. This typically involves recovery of the material wherein the terpenoid is present (e.g. a cell lysate or specific fraction thereof, the medium wherein the terpenoid is secreted) and subsequent purification of the terpenoid. Means that can be employed to this end are known to the skilled person.

It is to be understood that although particular embodiments, specific configurations as well as materials and/or molecules, have been discussed herein for cells and methods according to the present invention, various changes or modifications in form and detail may be made without departing from the scope and spirit of this invention. The following examples are provided to better illustrate particular embodiments, and they should not be considered limiting the application. The application is limited only by the claims.

### EXAMPLES

### Example 1: Generation of BY4742 Knockout Strains and Quantification of β-Amyrin

Starting from BY4742 (hereafter referred to as wild-type, WT), we created individual knockout strains for HRD1, PEP4, and PAH1. We decided to disrupt the genes through highly efficient HR-guided CRISPR/Cas9. To this end, we generated knockout vectors based on pCAS-ccdB, a vector combining the two main components of the CRISPR system, the single guide RNA (sgRNA) as well as the Cas9 nuclease on only one plasmid (Arendt et al 2017 Metabolic Engineering 40: 165-175).

As yeast favors to repair double strand breaks through homologous recombination, we designed homologous recombination donor oligonucleotides (HR donors) that integrated both a stop codon as part of a DraI restriction site as well as an additional or missing nucleobase to introduce a frameshift mutation. With this dual approach, we hoped to minimize the risk of suppressor mutations. We obtained individual knockouts for all three genes with efficiencies ranging between 40% and 94% (FIG. 1).

After curing the knockout strains from the URA3-containing pCAS vectors through counter selection on 5-fluoroorotic acid, we transformed the strains with pAG426GAL[GgbAS] for production of β-amyrin (FIG. 2A) and quantified the sequestered amounts of β-amyrin after five days of cultivation (FIG. 2B). Unexpectedly, the hrd1 strain did not exhibit an increased level of ergosterol and β-amyrin was produced to the same extent as in the wild-type strain. In contrast however, the pep4 and pah1 strains increased the accumulation of β-amyrin in the culture medium by ca. twofold and eightfold, respectively. This trend was also reflected by the levels of endogenous ergosterol, which accumulated about twofold and fourfold more in the pep4 and the pah1 strains, respectively. We consequently discarded the hrd1 strain as a potential candidate for triterpene over-production and continued with the remaining two knockout strains.

### Example 2: Effects of PEP4 and PAH1 Knockouts on the Production of Highly Oxygenated Sapogenins

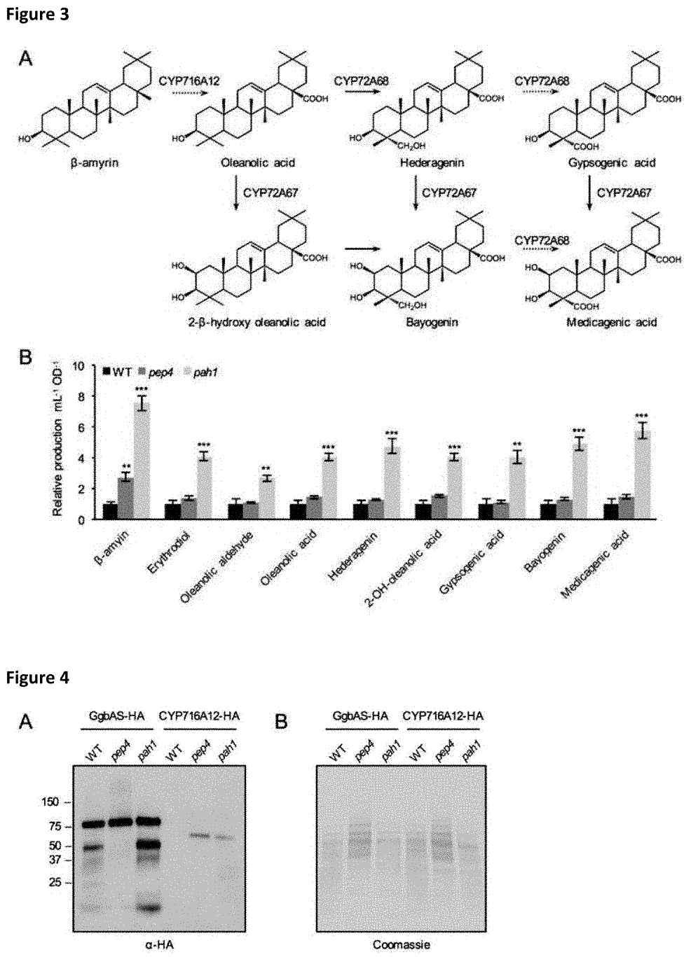

We subsequently assessed the capacities of the pep4 and pah1 strains for the production of higher oxidized sapogenins. To this end, we engineered a single plasmid that contains the genes of the Medicago truncatula medicagenic acid pathway, namely CYP716A12 for oxidation of C28 (Carelli, M. et al., Plant Cell, 2011), CYP72A68 for C23-oxidation (Fukushima, E. O. et al., Plant Cell Physiol., 2013), CYP72A67 for hydroxylation at C2 (Biazzi, E. et al., Mol. Plant, 2015), as well as the NADPH-cytochrome P450 reductase MtCPR1 (Arendt et al 2017 Metabolic Engineering 40: 165-175) (FIG. 3A). After super-transformation of the GgbAS-expressing strains with pESC-LEU[GAL1/MtCPR1-T2A-CYP716A12; GAL10/CYP72A67-E2A-CYP72A68], we quantified the amounts of sequestestered medicagenic acid and its intermediates after five days of cultivation. Strikingly, while retaining the same amount of β-amyrin as observed for the mere expression of GgbAS, the pep4 strain did not show an increased sapogenin production (FIG. 3B). In contrast, the knockout of PAH1 triggered an immense production boost as it resulted in the increased accumulation of medicagenic acid and its intermediates up to sixfold compared to the wild-type strain.

### Example 3: Analysis of Heterologous Expression Levels of Pep4 and Pah1 Strains

To analyze why the pep4 strain displayed a higher production capacity for β-amyrin while retaining the same amount of oxidized derivatives, we subsequently analyzed the recombinant gene expression levels at the protein level. Therefore, we expressed versions of GgbAS and CYP716A12 tagged with a C-terminal HA-peptide from the inducible, high copy number pAG426GAL plasmids and analyzed their expression levels via immunoblot. The pep4 strain accumulated more GgbAS protein than the wild-type and exhibited almost no protein degradation products, which were very abundant in the wild-type (FIG. 4A), pointing to vacuolar degradation of recombinant GgbAS protein in yeast. The pah1 strain also produced more GgbAS protein, albeit with a more pronounced spectrum of low-molecular degradation products. The increased protein production was more striking for CYP716A12, as for this construct no band could be detected in the wild-type, while similar amounts were present in both of the two knockout strains. Again, the pep4 strain lacked the degradation products that were present in the pah1 strain (FIG. 4A). As the increased accumulation of the P450 in peptidase-deficient pep4 strain should also result in a higher accumulation of oxidized triterpenoids, we concluded that the excess enzyme detected by immunoblot is probably misfolded and hence non-functional. The pah1 strain, however, likely has an increased capacity for the accommodation of functional ER-localized cytochromes P450, which is also reflected by increased production amounts on metabolite level. Due to the antagonistic effect of the PEP4 knockout in the pah1 background, we discarded PEP4 as a good gene target for the engineering of a generic triterpenoid overproducing yeast strain and focused on the pah1 strain for our following investigations.

### Example 4: Impact of the PAH1 Knockout on Growth Performance

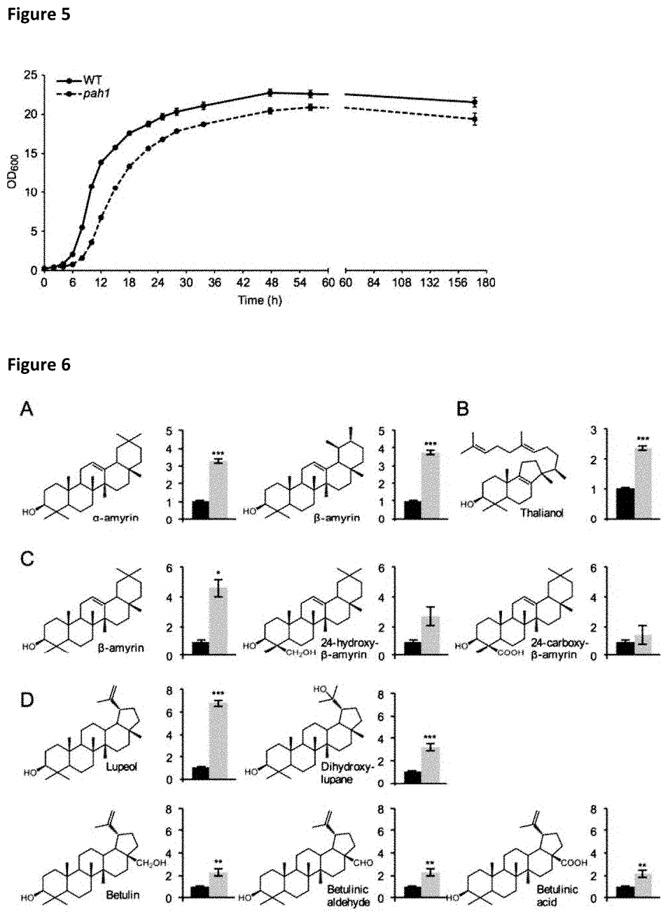

Yeasts strains with oversized ERs, as it is present for a deficient PAH1 gene, may show a growth retardation that can vary depending on the employed culture conditions. While glucose as main carbon source has only little impact, the growth is more severely impaired when other carbon sources such as galactose or fatty acids are used (Guerfal, M. et al., Microb. Cell Fact., 2013). Furthermore, pah1 strains display an increased sensitivity to elevated temperatures (Santos-Rosa, H. et al., EMBO J., 2005). Because metabolic engineering programs aim on the generation of robust microbial cell systems for the (over)production of target compounds, we verified the growth curve of our ER-engineered pah1 yeast. To this end, we cultured wild-type and knockout strains in 50 mL YPD in shake flasks and monitored the OD600 over a period of 170 h. In agreement with previous studies, the knockout showed a more pronounced lag phase compared to the wild-type, and reached only a slightly, not significantly, lower, final OD600 during stationary phase compared the wild-type (FIG. 5).

### Example 5: Effect of the PAH1 Knockout on the Production of Other Triterpenoid Classes

Subsequently, we wanted to determine whether the increased production capacity for medicagenic acid of the ER-engineered pah1 yeast can be translated to other triterpenoid classes. To this end, we expressed different OSCs alone or in combination with a P450 to produce structurally distinct triterpene skeletons. The effect could be confirmed for the OSC products α-amyrin and β-amyrin after expression of dammarenediol synthase from Centella asiatica (CaDDS) as well as for the three-ring structure thalianol after expression of thalianol synthase from A. thaliana (AtTHAS1) in combination with a truncated and de-regulated HMG1 (FIG. 6A+B). In both cases, the production was increased between two and fourfold. Co-expression of GgbAS with tHMG1 and CYP93E9 from Phaseolus vulgaris resulted in the increased production 24-hydroxy-β-amyrin and its acid (FIG. 6C). Furthermore, betulinic acid and its intermediates lupeol, betulin, betulinic aldehyde, betulinic acid as well as 3β,20-dihydroxylupane were significantly more abundant in spent culture medium of the pah1 strain after co-expressing lupeol synthase from A. thaliana (AtLUP1) with the C28-oxidase CYP716A83 from C. asiatica in combination and tHMG1 (FIG. 6D). We concluded that the effect of an engineered ER on the production levels is independent of the precise triterpene structure and can thus likely be applied to all triterpenoids.

### Example 6: Production of Triterpenoid Glucosides (Saponins)

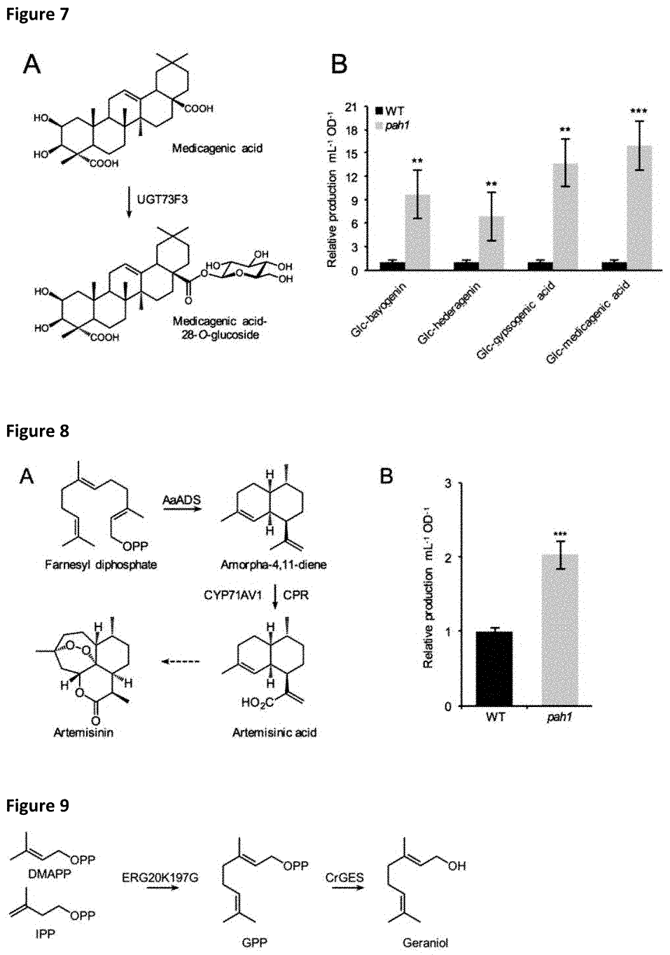

Many saponins exhibit bioactivities that make them interesting for pharmacological applications such as their use as vaccine adjuvant (Kensil, C., Crit. Rev. Ther. Drug Carrier Syst., 1996). The amphipathic properties are conveyed to the highly apolar triterpenoid skeletons by conjugation with sugar moieties through action of UDP-dependent glycosyltransferases (UGTs), thus generating glycosylated saponins (FIG. 7A). We therefore next investigated if the increased production capacity displayed by the pah1 strain can be translated to products that are further downstream in the saponin biosynthesis pathway. To this end, we expressed the M. truncatula UGT73F3 (Naoumkina, M. A. et al., Plant Cell, 2010) from pAG423GAL in the strains previously engineered for medicagenic acid production and quantified the titerpenoid-28-O-glucoside levels inside the cells after five days of cultivation. Notably, the ER-engineered pah1 strain showed a 16-fold production boost for medicagenic acid-28-O-glucoside, the final product of the pathway, thereby even exceeding the fold-increase observed for un-glycosylated sapogenins (FIG. 7B).

### Example 7: Production of Other Terpenoid Classes

Next, we analyzed the capacity of our ER-engineered pah1 yeast for the production of other classes of terpenoids. We chose to analyze the production of the sesquiterpenoid artemisinic acid.

Artemisinic acid is a precursor of the important anti-malarial drug artemisinin and can be produced in yeast via a two-step process from the universal sesquiterpene precursor FPP (Ro, D. K. et al., Nature, 2006) (FIG. 8A). To assess the effect of ER proliferation on the production of artemisinic acid, we followed the strategy of Ro et al. by expressing amorpha-4,11-diene synthase (AaADS) from pAG425GAL[AaADS] in combination with AaCYP71AV1 and AaCPR from pESC-URA[AaCYP71AV1/AaCPR] (Ro, D. K. et al., Nature, 2006). After five days of cultivation, the PAH1 knockout produced about twofold more artemisinic acid compared to the wild-type strain (FIG. 8B).

### Example 8: The Effect of ER Engineering on Production of Monoterpenoids

In this application we disclose that the modification of subcellular structures (using PAH1 as target) rather than the actual metabolic pathways or regulation thereof is a surprisingly effective tool for metabolic engineering. To our knowledge, this is the first report on the positive impact of the manipulation of subcellular structures on terpenoid production. Furthermore, we believe pah1 to be the biggest single KO effect ever reported for terpenoid engineering in yeast, resulting in an up to 16-fold improvement in the production of triterpene saponins, thereby greatly exceeding previously reported KO effects (Asadollahi et al. 2009 Metab Eng 11:328-334; Ignea et al. 2012 Microb Cell Fact 11:162; Özaydin et al. 2013 Metab Eng 15:174-183; Sun et al. 2014 Plos One 9, e112615; Trikka et al. 2015 Microb Cell Fact 14:60).

These findings disclosed in the application can be easily translated to other classes of terpenoids by the person skilled in the art. For example to demonstrate the impact of the pah1 mutation on monoterpenoid production, the skilled one can follow the strategy of Fischer et al. and transform the ERG20-deficient strain AE103 (Mata, his3Δ1, leu2Δ0, met15Δ0, ura3Δ0, ERG20::kanMX4; [pFL44ERG20]) with pAG424GAL[ERG20K197G] (FIG. 9). Yeast does not produce geranyl diphosphate (GPP), which is used as substrate of monoterpene synthases. Instead, yeast farnesyl diphosphate (FPP) synthase (encoded by ERG20) catalyzes the consecutive condensation of dimethyl allyl diphosphate (DMAPP) with two molecules of isopentenyl diphosphate (IPP). Several point mutations in ERG20 such as K197G were described that lead to a premature stop of DMAPP and IPP condensation, resulting in the production of both GPP and FPP. However, mere expression of such an ERG20 variant leads to only minimal amounts of free GPP which is in turn reflected in low monoterpene levels when co-expressed with a monoterpene synthase. It was demonstrated that expression of ERG20K197G in an erg20 yeast results in GPP levels sufficient for downstream reactions (Fischer, M. J. et al., Biotechnology and bioengineering, 2011). After obtaining transformants, the plasmid-borne copy of ERG20 can be cured through plating on 5-fluoroorotic acid-containing SD plates with tryptophan dropout, thus generating a starter strain with intracellular GPP pool for production of monoterpenoids. Using this starter strain, PAH1 should be knock out for example by employing the same CRISPR/Cas9-based strategy as described herein before. After super-transformation of the monoterpenoid starter strain and its pah1 derivative with for example pAG426GAL[CrGES] for expression of Catharanthus roseus geraniol synthase, both strains are cultivated for five days with a dodecane overlay to capture the produced monoterpenoid geraniol.

### Example 9: The Effect of ER Engineering on Production of Diterpenoids

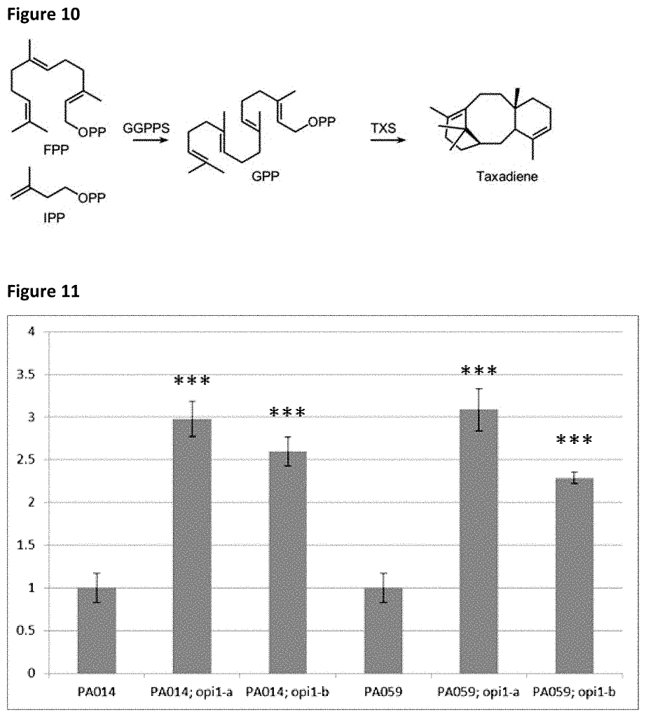

Similar as is described in Example 8, the pah1 mutation can be used to boost the production of diterpenoids, for example that of paclitaxel. Paclitaxel or Taxol® is probably the best known diterpenoid and is a potential drug for the treatment of various types of cancer. The first committed intermediate of the paclitaxel biosynthesis, taxadiene, is synthesized in a single enzymatic conversion from geranyl geranyl diphosphate (GGPP) (FIG. 10). Taxadiene was previously produced in S. cerevisiae through expression of Taxus chinensis taxadiene synthase (TcTXS). As intracellular levels of GGPP in yeast are low, however, additional expression of GGPP synthase (GGPPS) was shown to be essential for diterpenoid production in yeast (Engels, B. et al., Metabolic engineering, 2008). Following the same strategy as Engels et al., the skilled one can express TcTXS from pAG426GAL[TcTXS] and TcGGPPS from pAG425GAL[TcGGPPS] and compare taxadiene production in WT and pah1 cells.

### Example 10: Boosting the Production of Terpenoids Through Regulation of PAH1 Expression

On transcriptional level, PAH1 expression is known to be controlled by two regulatory complexes, the Ino2p/Ino4p/Opi1p regulatory circuit and the transcription factors Gis1p and Rph1p which bind to different positions of PPAH1 and as such induce gene expression. It was demonstrated that loss-of-function of either of the components leads to decreased PAH1 expression (Pascual, F. et al., Journal of Biological Chemistry, 2013). We decided to illustrate that overproduction of terpenoid production can not only be achieved by PAH1 knockout, but also through loss-of-function of regulatory units of PAH1. Starting from WT BY4742, we created individual knockout strains for OPI1 through HR-guided CRISPR/Cas9 as previously described for PAH1. After curing the knockout strains from the URA3-containing pCAS vectors through counter selection on 5-fluoroorotic acid, we transformed the strains with pAG426GAL[GgbAS] for production of β-amyrin and quantified the sequestered amounts of β-amyrin after five days of cultivation (FIG. 11). As in the case of the pah1 strains, increased accumulation of β-amyrin in the culture medium by ca. two- to three-fold was detected in all opi1 strains (FIG. 11).

### Example 11: Boosting the Production of Terpenoids Through Regulation of PAH1 Activity

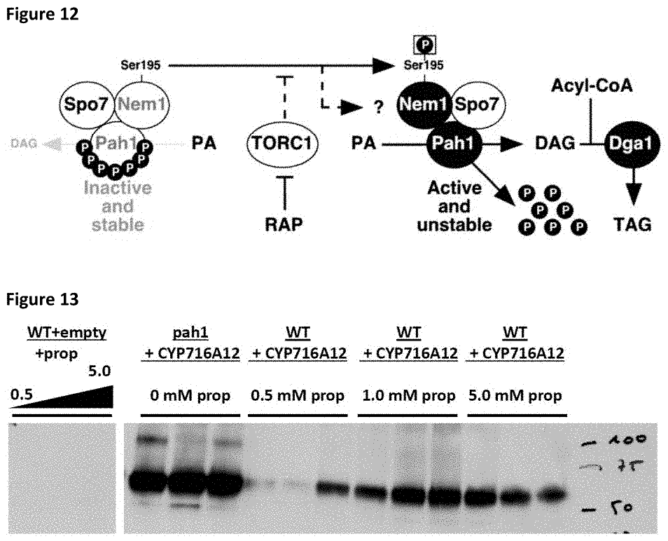

Pah1p activity is furthermore regulated on the protein level. To allow binding to the nuclear and ER membranes and as such initiate the PAP reaction, Pah1p needs to be dephosphorylated through action of the heterodimeric Nem1p/Spo7p phosphatase complex (FIG. 12). Knockout of either NEM1 or SPO7 was shown to dramatically reduce intracellular levels of neutral lipids in favor of phospholipid biosynthesis (Pascual, F. et al., Journal of Biological Chemistry, 2013). It was furthermore demonstrated that spo7 yeast cells exhibit a proliferation of the peripheral ER similar to that observed in pah1 cells (Campbell, J. L. et al., Molecular biology of the cell, 2006). In line with the findings disclosed herein, knocking-out Nem1 or Spo7 resulting in increased PAP activity increased the production of β-amyrin (data not shown).

Besides the Nem1p/Spo7p regulation of PAH1, it is known that several compounds inhibit PAP activity, more precisely propranolol (Brohée et al 2015 Oncotarget; Grkovich et al 2006 J Biol Chem; Albert et al 2008 J Leukoc Biol; Han and Carman 2010 J Biol Chem 285: 14628-14638); the sphingoid bases sphingosine and sphinganine (Han and Carman 2010); N-ethylmaleimide (NEM) (Grkovich et al 2006; Han and Carman 2010); bromoenol lacton (BEL) (Grkovich et al 2006; Albert et al 2008); rutin (Han et al 2016 Am J Ch Med 44: 565-578) and the HIF-1 inhibitors kaempferol and MTD (Mylonis et al 2012 J Cell Sc 125: 3485-3493). To analyze whether production of terpenoids can also be boosted through the use of the in the art known PAP activity inhibitors, we analyzed the impact of propranolol on the protein level of CYP716A12. The Western blot detecting CYP716A12 (FIG. 13) clearly show in WT that CYP716A12 levels increase with increasing concentration of propranolol. Note that in WT background CYP716A12 cannot be detected in the absence of propranolol. The CYP716A12 levels in WT upon addition of 1 mM propranolol mimicked that the CYP716A12 levels of untreated pah1 yeast. These results clearly show that also inhibition of PAH1 activity can be used to boost the production of terpenoids.

Materials and methods

### Chemicals

All chemicals and solvents were purchased from Sigma-Aldrich unless specified otherwise. The amorpha-4,11-diene standard was a kind gift from Filip Van Nieuwerburgh (Department of Pharmaceutics, Ghent University).

### Yeast Strains

A list of yeast strains used in this study can be found in Table 2.

TABLE 2
List of yeast strains used in this study.

| Strain | Description | Product | Source |
|---|---|---|---|
| BY4742 | MATa; his3Δ1; leu2Δ0; ura3Δ0; lys2Δ0 | — | EUROSCARF |
| PA005 | BY4742 hrd1 | — | This study |
| PA008 | BY4742 pep4 | — | This study |
| PA013 | BY4742; pESC-URA[GAL1/GgbAS; GAL10/tHMG1] | β-amyrin | This study |
| PA014 | BY4742; TRP1-Δ0; pMET3::ERG7 | β-amyrin | This study |
| PA032 | BY4742; pAG426GAL[GgbAS] | β-amyrin | This study |
| PA033 | PA005; pAG426GAL[GgbAS] | β-amyrin | This study |
| PA034 | PA008; pAG426GAL[GgbAS] | β-amyrin | This study |
| PA054 | BY4742; pAG426GAL[CYP716A12]-HA | — | This study |
| PA055 | BY4742; pAG426GAL[GgbAS]-HA | — | This study |
| PA057 | PA008; pAG426GAL[CYP716A12]-HA | — | This study |
| PA058 | PA008; pAG426GAL[GgbAS]-HA | — | This study |
| PA059 | BY4742 trp1 | — | (Karel Miettinen |
| | | | et al., 2016) |
| PA085 | BY4742; pAG425GAL[AaADS]; pESC-URA[AaCYP71AV1/AaCPR] | Artemisinic acid | This study |
| PA086 | PA008; pAG425GAL[AaADS]; pESC-URA[AaCYP71AV1/AaCPR] | Artemisinic acid | This study |
| PA186 | BY4742 pah1 | — | This study |
| PA188 | PA186; pAG426GAL[GgbAS]-HA | — | This study |
| PA189 | PA186; pAG426GAL[CYP716A12]-HA | — | This study |
| PA195 | PA186; pAG425GAL[AaADS]; pESC-URA[AaCYP71AV1/AaCPR] | Artemisinic acid | This study |
| PA199 | PA032; pESC-LEU[GAL1/MtCPR1-T2A-CYP716A12; | Medicagenic acid | This study |
| | GAL10/CYP72A67-E2A-CYP72A68] | | |
| PA200 | PA033; pESC-LEU[GAL1/MtCPR1-T2A-CYP716A12; | Medicagenic acid | This study |
| | GAL10/CYP72A67-E2A-CYP72A68] | | |
| PA201 | PA034; pESC-LEU[GAL1/MtCPR1-T2A-CYP716A12; | Medicagenic acid | This study |
| | GAL10/CYP72A67-E2A-CYP72A68] | | |
| PA202 | PA186; pAG426GAL[GgbAS] | β-amyrin | This study |
| PA338 | PA202; pESC-LEU[GAL1/MtCPR1-T2A-CYP716A12; | Medicagenic acid | This study |
| | GAL10/CYP72A67-T2A-CYP72A68] | | |

Construction of the Advanced CRISPR Vector pCASmGG

To facilitate the cloning of protospacer sequences for CRISPR/Cas9 experiments, we adapted the vector pCAS-ccdB (Miettinen, K. et al., Nat. Commun., Submitted, 2016) for GoldenGate cloning. To this end, the three SapI recognition sites in the vector backbone were mutagenized by PCR-amplification of pCAS1 using primer pairs P1+P2, P3+P4, and P5+P6, each designed to mutagenize SapI recognition sites. The three fragments were SapI treated, gel-purified, and ligated, thereby yielding pCASm. Subsequently, pCASm-ccdB was generated by cloning the Gateway™ ccdB cassette into pCASm as described previously (Miettinen, K. et al., Nat. Commun., Submitted, 2016). The gRNA-GoldenGate cassette was generated by PCR-amplifying SNR52p and crRNA-SUP4t from p426-SNR52p-gRNA.CAN1.Y-SUP4t (Dicarlo, J. E. et al., Nucleic Acids Res., 2013) using primer pairs P7+P9 and P10+P8. A lacZ cassette was PCR-amplified from pICH47751 (addgene #48002) using primers P11 and P12. Then, the three fragments were joined by overlap extension PCR, sub-cloned into pDONR221, and finally recombined into pCASm-ccdB, thereby creating pCASmGG.

Cloning of Protospacer Sequences into pCAS-ccdB and pCASmGG

For the knockout of PEP4 and HRD1, sgRNA cassettes were cloned using primers P7+P8, respectively, as described previously (Miettinen, K. et al., Nat. Commun., Submitted, 2016). Functional CRISPR plasmids based on the advanced CRISPR vector pCASmGG were generated by GolgenGate cloning of short fragments comprising the protospacer sequences with a 5′ ATC and a 3′ TAA overhang. Fragments were made by heating a solution of two oligonucleotides at a concentration of 20 μM in 1× buffer C (Promega) in a boiling water bath for 5 min and slowly cooling down to room temperature. The solution was diluted 100-fold in 1× buffer C and 1 μM was used for a standard GoldenGate reaction with 100 ng pCASmGG, T4 ligase (Thermo Fisher Scientific), and the type IIS restriction enzyme SapI (New England Biolabs). The reaction mixture was incubated at 20° C. and 37° C. for 5 min each for 30 cycles with final incubations at 50° C. and 80° C. for 10 min each. The reaction mixture was used to transform E. coli cells that were subsequently plated on LB plates containing appropriate amounts of carbenicillin, IPTG, and X-Gal. Positive colonies were identified by blue-white selection and selected plasmids were analyzed by control digest and Sanger sequencing. Oligonucleotides used for cloning are listed in table 1.

TABLE 1
List of oligonucleotides used in this study

| | | |
|---|---|---|
| Name | Sequence | Description |

Primers used for the construction of pCASmGG

| | | |
|---|---|---|
| P1 | ACAAAAgctcttcCGCTTTTGGCAATGTCAACAGTACCCTTAGT | pCAS domestication 1 Fw |
| P2 | TATATAgctcttcGCGCTTCCTCGCTCACTGACT | pCAS domestication 1 Rv |
| P3 | CAGTGAgctcttcAGCGCTAGAGCGCCCAATACGCAAAC | pCAS domestication 2 Fw |
| P4 | CGCCAGgctcttcTCAAATTTCGGGGACACTTCCT | pCAS domestication 2 Rv |
| P5 | AGTGTCgctcttcTTTGATCATATGCGCCAGCG | pCAS domestication 3 Fw |
| P6 | TATATAgctcttcGAGCGACAAAGATTTTGTTATCGG | pCAS domestication 3 Rv |
| P7 | GGGGACAAGTTTGTACAAAAAAGCAGGCTTAAAGGGAACAAAAGCTGGAGC | attB1-P_{SNR52} Fw |
| P8 | GGGGACCACTTTGTACAAGAAAGCTGGGTAAAAGCCTTCGAGCGTCCC | attB2-T_{cyc1} Rv |
| P9 | GACAAGCTGTGACGCTCTTCGGATCATTTATCTTTCACTGCGGAGA | P_{SNR52} lacZ part Rv |
| P10 | GTGCCAGCTGCGCTCTTCGGTTTTAGAGCTAGAAATAGCAAGTTAAAATAAGG | gRNA lacZ part Fw |
| P11 | CTCCGCAGTGAAAGATAAATGATCCGAAGAGCGTCACAGCTTGTCTGTAAGCGGAT | lacZ Fw |
| P12 | ATTTCTAGCTCTAAAACCGAAGAGCGCAGCTGGCACGACAGG | lacZ Rv |

Primers for cloning of gRNA cassettes into pCAS-ccdB

| | | |
|---|---|---|
| P13 | ACCAAGTTGCTGCAAAAGTCGATCATTTATCTTTCACTGCGGAGAAG | PEP4 left Rv |
| P14 | GACTTTTGCAGCAACTTGGTGTTTTAGAGCTAGAAATAGCAAGTTAAAATAAGG | PEP4 right Fw |
| P15 | TACTATGGCAACTCCTAACGATCATTTATCTTTCACTGCGGAGAAG | HRD1 left Rv |
| P16 | GTTAGGAGTTGCCATAGTAGTTTTAGAGCTAGAAATAGCAAGTTAAAATAAGG | HRD1 right Fw |

Oligos for protospacer cloning in pCASmGG

| | | |
|---|---|---|
| P17 | atcGATTGAGGGGGGCTTGATG | PAH1 Fw |
| P18 | aacCATCAAGCCCCCCTCAATC | PAH1 Rv |

HR donor oligonucleotides

| | | |
|---|---|---|
| P19 | TTGTCGATATTCATCTTATTAAATTCTTAATACTATGGCAACTCCTAACGAAACTATT | HRD1 HR Fw |
| P20 | AATAGTTTCGTTAGGAGTTGCCATAGTATTAAGAATTTAATAAGATGAATATCGACAA | HRD1 HR Rv |
| P21 | ATTGGCCTTGTTGTTGGTCAGCTAAAACCAAGTTtttaaaAGTCCACAAGGCTAAAA | PEP4 HR Fw |
| P22 | TTTTAGCCTTGTGGACTtttaaaAACTTGGTTTTAGCTGACCAACAACAAGGCCAAT | PEP4 HR Rv |
| P23 | ATTGCTTGTGTCGCCCGTGATGAGCGtttaaaTCAAGCCCCCCTCAATCACCTGAAACA | PAH1 HR Fw |
| P24 | TGTTTCAGGTGATTGAGGGGGGCTTGAtttaaaCGCTCATCACGGGCGACACAAGCAAT | PAH1 HR Rv |

Primers used for RFLP analysis

| | | |
|---|---|---|
| P25 | GGTGCCAGAAAATAGAAGGAAAC | HRD1 RFLP Fw |
| P26 | GTTGTGAGCGATAAAATTCCCAG | HRD1 RFLP Rv |
| P27 | TTCATTTGCGGGTGTCGATG | PEP4 RFLP Fw |
| P28 | GGAACATCGTGACCACCTTC | PEP4 RFLP Rv |
| P29 | TCCAGACGGAAGGCTATCATG | PAH1 RFLP Fw |
| P30 | ATGTTGCCATTTTTGTCCTCC | PAH1 RFLP Rv |

Primers used for generation of expression vectors

| | | |
|---|---|---|
| P31 | TATATAgcggccgcATGCCAACTTTGTACAAAAAAGCAG | Notl-CYP72A67 Fw |
| P32 | CAATTTCAAGAGAGCATAATTAGTACACTGTCCGCTTCCTGCTTTCACTTTGCGTAG | CYP72A67-E2A Rv |
| P33 | TGCTCTCTTGAAATTGGCTGGAGATGTTGAGAGCAACCCTGGACCTATGGAATTAT | E2A-CYP72A68 Fw |
| | CTTG | |
| P34 | TATATAagatctTGTACAAGAAAGCTGGGTATTATGT | Bg/ll-CYP72A68 Rv |
| P35 | TATATAgtcgacATGACTTCTTCCAATTCCGATTTAGT | Sa/l-MtCPR1 Fw |
| P36 | CGTCACCGCATGTTAGCAGACTTCCTCTGCCCTCCCAGACATCCCTAAGGTAGCG | MtCPR1-T2A Rv |
| P37 | CTGCTAACATGCGGTGACGTCGAGGAGAATCCTGGCCCAATGGAGCCTAATTTC | T2A-CYP716Al2 Fw |
| P38 | TATATActcgagTTAAGCTTTGTGTGGATAAAGGCG | Xhol-CYP716Al2 Rv |

Gateway ™ recombination sites are underlined and lower case letters represent either restriction enzyme recognition sites or base overhangs after oligonucleotide annealing.

### Construction of Expression Vectors

Expression vectors were mostly generated by Gateway™-recombination of entry clones available in-house with destination vectors (Alberti, S. et al., Yeast, 2007), addgene Kit #1000000011). For the construction of the vector expressing the M. truncatula medicagenic acid genes as self-splicing polyproteins, first CYP72A67 and CYP72A68 were PCR-amplified from plasmid DNA using primer pairs P31+P32 and P33+P34. The fragments were joined by overlap extension PCR using primers P31+P34. Both the fragment and pESC-LEU (Agilent) were cut with NotI and BgIII (Promega) and ligated using T4 ligase (Thermo Fisher), thereby generating pESC-LEU[GAL1/CYP72A67-E2A-CYP72A68]. Then, MTR1 and CYP716A12 were amplified from plasmid DNA using primer combinations P35+P36 and P37+P38, respectively. Fragments were joined using primers P35+P38, cut with SalI and XhoI (Promega) and cloned into the intermediate vector. The resulting vector, pESC-LEU[GAL1/MtCPR1-T2A-CYP716A12; GAL10/CYP72A67-E2A-CYP72A68], was verified by Sanger sequencing and control digest.

### Yeast Transformation

Transformations were done following the standard lithium acetate/single-stranded carrier DNA/polyethylene glycol method (Gietz, R. D. and Woods, R. A., Methods in Enzymology, 2002).

### CRISPR-Mediated Gene Knockout

The gene knockout in S. cerevisiae was carried out as described previously (Miettinen, K. et al., Nat. Commun., Submitted, 2016) and HR recombination donors are listed in table 1. The analysis of the resulting clones was done by yeast colony PCR and positive clones were identified upon RFLP. Positive HRD1 knockouts resulted in a loss of a BsII (New England Biolabs) restriction site and all other knockouts were identified by the gain of a DraI (Promega) site. Positive knockout events were confirmed by Sanger sequencing of the respective locus and the pCAS vectors were cured by plating the strains on YPD plates containing 1 mg mL-1 5-FOA (Zymo Research).

### Growth Curve

For the recording of the growth curve, five independent pre-cultures were grown in 5 mL YPD in cultivation tubes at 30° C. and 250 min-1 for 48 h to allow all cells to reach the same developmental stage and subsequently diluted to an OD600 of 0.25 in 50 mL YPD in a 250 mL culture flask. The cultures were continued to grow under the same condition and the OD600 was periodically measured using appropriate dilutions of the culture medium.

### Immunoblot

For immunoblot analysis, pre-cultures were grown in 5 mL SD-Ura (Duchefa, Clontech) at 30° C. and 250 min-1 for 24 h. Cells were then sedimented by centrifugation, washed with 5 mL sterile water and used to inoculate 50 mL SD-Gal/Raf-Ura in a 250 mL culture flask to an OD600 of 0.1 for induction of protein synthesis. Cells were grown for another 24 h using the same culturing parameters. Total protein was extracted following standard procedure and 30 μg of total protein was separated by SDS-PAGE (4-15% Mini-PROTEAN® TGX™ Precast Gel, Bio-Rad) and blotted on a polyvinylidene fluoride (PVDF) membrane (Trans-Blot® Turbo™ Mini PVDF Transfer; Bio-Rad). After incubation with anti-HA High Affinity (Sigma-Aldrich) and anti-rat-horseradish peroxidase (Sigma-Aldrich), the signal was captured using detection substrate (Western Lightning® Plus-ECL; Perkin Elmer) and X-ray films (Amersham Hyperfilm ECL; GE Healthcare). Total protein loading was visualized using Coomassie Brilliant Blue (Thermo Scientific) staining of the PVDF membrane.

### Culturing of Yeast Cells for Metabolite Analysis

For all production cultures, precultures were grown at 30° C. and 250 min-1 for 48 h in 5 mL synthetic defined (SD) medium with glucose (Duchefa, Clontech) and appropriate amino acid and nucleobase drop out (DO) supplements (Duchefa, Clontech). Yeast cells were collected by centrifugation, washed in 5 mL sterile water and subsequently induced for metabolite production.

### Production and Quantification of Triterpenoids

Precultures were resuspended in 5 mL SD Gal/Raf medium (Duchefa, Clontech) supplemented with 10 mM MβCD and cultivated for 5 days. For metabolite analysis, 50 μL of a 0.1 mg mL-1 solution of cholesterol in ethanol was added as internal standard to 1 mL culture medium, which then was extracted with 700 μL ethyl acetate. The extract was evaporated under vacuum and the sample derivatized by addition of 10 μL pyridine and 50 μL N-Methyl-N-(trimethylsilyl)trifluoroacetamide. The GC analysis was performed as previously described (Moses, T. et al., Proc. Natl. Acad. Sci. U.S.A, 2014). Quantification was carried out on the areas of the full-MS scan. Due to co-elution with a contaminant, oleanolic aldehyde was quantified based on the oleanane-type sapogenin-specific ion 203 m/z. For the analysis of cultures producing triterpenoid glucosides (saponins), cells from 2 mL production medium were frozen in liquid nitrogen and disrupted using a bead mill (Retsch) with one metal bead of 5 mm and two beads of 3 mm diameter for 2 min and 25 Hz. The pellet was extracted with 1 mL methanol. After evaporation of the solvent, the sample was counter-extracted with 150 μL water and 150 μL cyclohexane. The water phase was analyzed via LC-MS.

### Production and Quantification of Artemisinic Acid

The production cultures were prepared as described for the production of triterpenoids with exception of the supplementation with MβCD. After 5 days of cultivation, cells corresponding to 5 mL culture medium were extracted as described elsewhere (Ro, D. K. et al., Nature, 2006). In brief, the cells were thoroughly washed with 5 mLTris/HCl pH 9, the buffer was acidified to pH 2 and was extracted with 1 mL ethyl acetate spiked with 1 μg mL-1 8-octyl benzoic acid. The solvent was evaporated under vacuum, derivatized as described above and analyzed by GC-MS. The injector temperature was set to 280° C. and the oven held at 70° C. for 1 min after injection. The temperature was ramped to 210° C. at an increment of 5° C. min-1, held for 5 min, ramped to 320° C. at 20° C. min-1, held for 1 min, and eventually decreased to 80° C. at 50° C. min-1 and held for 2 min. The MS settings used were the same as used for the analysis of triterpenoids with a solvent delay of 11 min. The internal standard was quantified based on peak areas of 119 m/z and artemisinic acid for peaks of 216 m/z as compared to an authentic standard.

### LC-MS Analysis

For LC-MS, 10 μL of the sample was injected in a ZORBAX RRHD Eclipse XDB-C18 column (2.1×150 mm, 1.8 μm) (Strictosidine) or an Acquity UPLC BEH C18 column (2.1×150 mm, 1.7 μm) (Triterpene glycosides) mounted on a Thermo instrument equipped with an LTQ FT Ultra and an electrospray ionization source. The following gradient was run using acidified (0.1% (v/v) formic acid) solvents A (water/acetonitrile, 99:1, v/v) and B (acetonitrile/water; 99:1, v/v): time 0 min, 5% B; 30 min, 55% B; 35 min, 100% B. Triterpene glycosides were analyzed in negative ionization mode with the following parameter values: capillary temperature 150° C., sheath gas 25 (arbitrary units), aux. gas 3 (arbitrary units) and spray voltage 4.5 kV. For identification, full MS spectra were interchanged with a dependent MS2 scan event in which the most abundant ion in the previous full MS scan was fragmented, two dependent MS3 scan events in which the two most abundant daughter ions were fragmented and a dependent MS4 scan event in which the most abundant daughter ion of the first MS3 scan event was fragmented. The collision energy was set at 35%.

1. A recombinant eukaryotic cell, the recombinant eukaryotic cell comprising:
at least one chimeric gene construct comprising
a promoter active in the recombinant eukaryotic cell operably fused to a nucleic acid encoding a terpenoid biosynthesis enzyme;
wherein intracellular membrane proliferation is increased in the recombinant cell in comparison with a control cell.

2. The recombinant eukaryotic cell of claim 1, wherein negative regulation of intracellular membrane proliferation is inhibited.

3. The recombinant eukaryotic cell of claim 1, wherein expression and/or activity of an endogenous phosphatidic acid phosphatase is inhibited in the recombinant eukaryotic cell, and/or wherein the eukaryotic cell overexpresses a diacylglycerol kinase or has an increased activity of an endogenous diacylglycerol kinase in the recombinant eukaryotic cell.

4. The recombinant eukaryotic cell of claim 3, wherein the endogenous phosphatidic acid phosphatase is PAH1 and/or wherein the diacylglycerol kinase is DGK1.

5. The recombinant eukaryotic cell of claim 1, wherein the cell is a yeast cell.

6. A cell culture comprising the recombinant cell of claim 1.

7. A method for the production of a terpenoid in a recombinant eukaryotic cell, the method comprising:
providing a eukaryotic cell wherein the intracellular membrane proliferation is increased in the cell in comparison with a control cell,
introducing into the eukaryotic cell at least one chimeric gene construct comprising a promoter active in the eukaryotic cell operably fused to a nucleic acid sequence encoding a terpenoid biosynthesis enzyme so as to produce the recombinant eukaryotic cell; and
culturing the recombinant eukaryotic cell in conditions suitable for producing the terpenoid.

8. The method according to claim 7, wherein negative regulation of intracellular membrane proliferation is inhibited in the recombinant eukaryotic cell.

9. The method according to claim 7, wherein expression and/or activity of an endogenous phosphatidic acid phosphatase is inhibited in the recombinant eukaryotic cell, and/or wherein a diacylglycerol kinase is overexpressed or has an increased activity of an endogenous diacylglycerol kinase in the recombinant eukaryotic cell.

10. The method according to claim 9, wherein said endogenous phosphatidic acid phosphatase is PAH1 and/or wherein the diacylglycerol kinase is DGK1.

11. The method according to claim 7, wherein the terpenoid is selected from the group consisting of hemiterpenoids, monoterpenoids, sesquiterpenoids, diterpenoids, sesterpenoids, triterpenoids, tetraterpenoids, polyterpenoids, and glycosides thereof.

12. The method according to claim 7, wherein the eukaryotic cell is a yeast cell.

13. The method according to claim 7, further comprising isolating the produced terpenoid.

14. (canceled)

15. (canceled)

16. The recombinant eukaryotic cell of claim 3, wherein the activity of an endogenous phosphatidic acid phosphatase is inhibited by a PAH1 inhibitor.

17. The recombinant eukaryotic cell of claim 16, wherein the PAH1 inhibitor is selected from the list consisting of propranolol, sphingosine, sphinganine, rutin, kaempferol, N-ethylmaleimide and bromoenol lactone.

18. The recombinant eukaryotic cell of claim 5, wherein the yeast cell is a S. cerevisiae cell.

19. The method according to claim 12, wherein the yeast cell is a S. cerevisiae cell.

20. The method according to claim 9, wherein the activity of an endogenous phosphatidic acid phosphatase is inhibited by a PAH1 inhibitor

21. The method according to claim 21, wherein the PAH1 inhibitor is selected from the list consisting of propranolol, sphingosine, sphinganine, rutin, kaempferol, N-ethylmaleimide and bromoenol lactone.
